SEQUENCE LISTING (14 sequences)

SEQ ID NO: 1 - protein, 303 aa, Mycobacterium tuberculosis
MSETPRLLFVHAHPDDESLSNGATIAHYTSRGAQVHVVTCTLGEEGEVIGDRWAQLTADHADQLGGYRIGELTAALRALGVSAPIYLGGAGRWRDSGMAGTDQRSQRRFVDADPRQTVGALVAIIRELRPHVVVTYDPNGGYGHPDHVHTHTVTTAAVAAAGVGSGTADHPGDPWTVPKFYWTVLGLSALISGARALVPDDLRPEWVLPRADEIAFGYSDDGIDAVVEADEQARAAKVAALAAHATQVVVGPTGRAAALSNNLALPILADEHYVLAGGSAGARDERGWETDLLAGLGFTASGT

SEQ ID NO: 2 - protein, 100 aa, Mycobacterium tuberculosis
MSETPRLLFVHAHPDDESLSNGATIAHYTSRGAQVHVVTCTLGEEGEVIGDRWAQLTADHADQLGGYRIGELTAALRALGVSAPIYLGGAGRWRDSGMAG

SEQ ID NO: 3 - protein, 8 aa, Artificial sequence
  Domain of conservation
VHAHPDDE

SEQ ID NO: 4 - protein, 11 aa, Artificial sequence
  Domain of conservation
VTCTLGEXGEV

SEQ ID NO: 5 - protein, 12 aa, Artificial sequence
  Domain of conservation
YDPXGGYGHPDH

SEQ ID NO: 6 - protein, 8 aa, Artificial sequence
  Domain of conservation
AXXAHATQ

SEQ ID NO: 7 - DNA, 909 bp, Mycobacterium tuberculosis
atgtctgagacgccgcggctgctgtttgttcatgcacaccccgacgatgagagcctgagcaacggcgcaaccatcgcgcactacacctcccgtggcgcacaggtccatgtcgtcacgtgcaccctgggtgaggagggcgaggtcattggcgatcgctgggctcaactcaccgccgatcatgcggaccaactcggtggctaccgcatcggcgagctcaccgcggcgttgcgagcgctcggggtcagcgcaccgatctaccttggcggcgcgggtcgctggcgcgactccggcatggccggcacagaccagcggagtcagcggagattcgtcgatgctgacccccggcagaccgtcggggcattggtcgcgatcattcgcgagctgcggccgcatgtcgtggtgacctatgaccccaatggcggttacggtcatcctgaccacgtgcacacccacaccgtcactaccgccgcggtggccgcagcgggtgttgggtccggtaccgcagatcaccccggcgacccgtggacggtgccgaagttctactggacggtcttgggtctgagcgcgctcatttcgggcgcgcgagccctggtccccgacgatctgcgacccgaatgggtgttgccgcgggccgacgagattgcattcgggtactccgacgacggtatcgacgccgtcgtcgaggccgatgagcaggcgcgagccgccaaggttgcggcactggctgcccatgccacccaagttgtcgtcggcccgaccggccgggccgccgccttgtcgaacaacctggcactgcccatcctggccgatgagcattacgtgctcgccggcggctccgcgggcgcccgcgatgaacgtggctgggaaactgatctgctcgccggtctgggcttcaccgcgtccggcacg

SEQ ID NO: 8 - DNA, 25 bp, Artificial sequence
  PCR primer
tagccatggtgtctgagacgccgcg

SEQ ID NO: 9 - DNA, 23 bp, Artificial sequence
  PCR primer
ggatcccgcggtgaagcccagac

SEQ ID NO: 10 - DNA, 23 bp, Artificial sequence
  PCR primer
ttcatatggtgtctgagacgccg

SEQ ID NO: 11 - DNA, 24 bp, Artificial sequence
  PCR primer
ataagcttgtagccggacgcggtg

SEQ ID NO: 12 - protein, 308 aa, Mycobacterium leprae
MSETPRLLFVHAHPDDESLSNGATIAHYTSRGAQVQVVTCTLGEEGEVIGDRWAELTVDHADQLGGYRIFELTEALRALGVSAPIYLGGAGRWRDSGMRGTAPRRRQRFIDADENEAVGALVAIIRELRPHVVVTYDPHGGYGHPDHVHTHFITAAAVASSGVAAGLEVGADEYPGKPWKVPKFYWSVFALSAFEAGMNALQGKDLRPEWTIPPREEFYFGYSDKDIDAVVEATSDVWAAKTAALTAHATQVVVGPTGRACALSNNMVMPIFAQEHYVLAAGSAGNRDERGWETDLLAGLCLDGFDAR

SEQ ID NO: 13 - protein, 297 aa, M. avium
MAETPRLLFVHAHPDDESLGTGATIAHYTAAGADVRVVTCTLGEEGEVIGERWAELAVDRADQLGGYRIGELTAALRELGVGEPCYLGGAGRWRDSGMPGTPKRRRQRFIDADEREAVGALVAVIREQRPHVVVGYDPAGGYGHPDHVHVHTVTTAAVAAAGAGDFPGEPWAVPKFYWSVFATRPFEAAVQALTPEDLRPDWSMPSAEQFTFGYADDHIDAVVAAGPHAWAAKRAALAAHATQVVVGPTGRACALSNNVALPILDEEHYVLVAGAAGARDERGWETDLLAGLEFGAA

SEQ ID NO: 14 - protein, 290 aa, M. smegmatic
MSSHESPRLLFVHAHPDDETLTTGGTIAHYVARSAEVHVVTCTLGEEGEVIGERYAQLAVDHADQLGGYRIAELTAALQSLGLRGPRYLGGAGHWRDSGMAGTPSRGRQRWVDADLDEAVGALVAVIGEVRPHVVVTYDPNGGYGHPDHIQTHVVTTRAVAAAPEAVGWTVPKFYWTVTAISAMTAGLQALGDVPSEWIRVNAEDIPFGFGDDQIDAVVDVTAELPAKVGAMRAHATQITVAPDGRAFALSNNIALPVLGEEHYVLVSGEAGPRDSRGWETDLLAGLDLE

(19) United States
(12) Patent Application Publication
(10) Pub. No.: US 2005/0124021 A1
(43) Pub. Date: Jun. 9, 2005

(54) Acetyl glucosaminyl inositol deacetylase, a mycothiol biosynthetic enzyme, and methods of use

(76) Inventors: Gerald L Newton, San Diego, CA (US); Jossef Av-Gay, Vancouver (CA); Robert C. Fahey, Del Mar, CA (US)

Correspondence Address: Lisa A Haile;Gray Cary Ware & Freidenrich, Suite 1100, 4365 Executive Drive, San Diego, CA, 92121, US

(21) Appl. No.: 10/297,512

(22) PCT Filed: Jun. 14, 2001
(86) PCT No.: PCT/US01/19091

Related U.S. Application Data
(60) Provisional application No. 60/211,612, filed on Jun. 14, 2000.

(51) Int. Cl. C12Q 1/34; C07H 21/04; C12Q 1/04; C12N 15/74; C12N 9/18

(52) U.S. Cl. 435/18; 435/34; 435/69.3; 435/197; 435/252.3; 435/320.1; 514/509; 536/23.2; 530/388.26

(57) ABSTRACT

The present invention provides a family of bacterial acetyl glucosaminyl inositol deacetylases (MshB) with deacetylase activity against acyl glucosaminyl inositol and which play a key role in mycothiol biosynthesis. The invention deacetylases are characterized by a conserved 100 amino acid N-terminal region and three highly conserved histidine-containing regions and by having deacetylase activity as well as amide hydrolase activity. The invention further provides methods for using the invention deacetylases in drug screening assays to determine compounds that inhibit activity. The invention provides for treatment of actino-mycete infections in mammals using antibiotics that inhibit production or activity of MshB and thereby reduce the production of mycothiol and the virulence of the infecting bacteria.

### FIELD OF THE INVENTION

The present invention generally relates to a family of enzymatic compounds produced by bacteria and methods of their use in drug discovery and disease control, and more specifically to acetyl glucosaminyl inositol deacetylases and methods of their use.

### BACKGROUND INFORMATION

Glutathione (GSH) is the dominant low molecular weight thiol in most eukaryotes and Gram-negative bacteria, and it plays a key role in protection of the cell against oxygen toxicity and electrophilic toxins. However, most gram positive bacteria, including many strict aerobes, do not produce glutathione. Yet aerobic organisms are subjected to oxidative stress from many sources, including atmospheric oxygen, basal metabolic activities, and, in the case of pathogenic microorganisms, toxic oxidants from the host phagocytic response intended to destroy the bacterial invader.

Actinomycetes, including Streptomyces and Mycobacteria, do not make GSH but produce instead millimolar levels of mycothiol (MSH, AcCys-GlcN-Ins), an unusual conjugate of N-acetylcysteine (AcCys) with 1-D-myo-inosityl-2-amino-2-deoxy-α-D-glucopyranoside (GlcN-Ins). The biochemistry of mycothiol appears to have evolved completely independently of that of glutathione. However, it has already been established that the metabolism of mycothiol parallels that of glutathione metabolism in two enzymatic processes. First, formaldehyde is detoxified in glutathione-producing organisms by NAD/glutathione-dependent formaldehyde dehydrogenase (L. Uotila, et al. (1989) in Glutathione: Chemical, Biochemical, and Medical Aspects—Part A (D. Dolphin, et al., Eds.) pp 517-551, John Wiley & Sons, et al.). An analogous process involving NAD/mycothiol-dependent formaldehyde dehydrogenase has been identified in the actinomycete Amycolatopsis methanolica (M. Misset-Smits, et al. (1997) FEBS Lett. 409:221-222). This enzyme has been sequenced (A. Norin, et al. (1997) Eur. J. Biochem. 248:282-289).

The second enzymatic process involves a mycothiol homolog of glutahione reductase recently cloned from M. tuberculosis and expressed in M. smegmatis (M. P. Patel, et al. (1999) J. Amer. Chem. Soc. 120:11538-11539; M. P. Patel et al. (1999) Biochemistry 38:11827-11833; M. P. Patel et al (2001) Biochemistry 40:3119-3126). The reductase is reasonably specific for the disulfide of mycothiol, but is also active with the disulfide of AcCys-GlcN, the desmyo-inositol derivative of mycothiol.

Therefore, there is a need in the art for methods and compounds useful for investigation of the details of the metabolism of mycothiol and comparison with the established roles for the metabolism of glutathione.

Antibiotic resistance of pathogenic bacteria, including pathogenic actinomycetes, such as M. tuberculosis, is a well-known problem faced by medical practitioners in treatment of bacterial diseases. Therefore, there is a need in the art for new antibiotics, drugs and vaccines and for screening techniques to discover antibiotics, drugs and vaccines effective to treat or prevent bacterial infections in humans and in other mammals, such as domestic and farm animals.

### SUMMARY OF THE INVENTION

The present invention solves these and other problems in the art by providing, in one embodiment, purified acetyl glucosaminyl inositol deacetylases, characterized as having an N-terminal region with an amino acid sequence with 40% or more sequence identity to SEQ ID NO:2 and conservative variations thereof, four domains of conservation, wherein three of the domains contain conserved histidine residues, and deacetylase activity against acetyl glucosaminyl inositol.

In another embodiment, the invention provides purified acetyl glucosaminyl inositol deacetylases characterized as having an N-terminal region with an amino acid sequence with at least 40% sequence identity to SEQ ID NO:2, one or more domains of conservation containing conserved metal chelating residues, and deacetylase activity against acetyl glucosaminyl inositol in the presence of metal ion. Antibodies and functional fragment thereof, that bind specifically to invention deacetylases, isolated polynucleotide that encode the invention deacetylases, vectors containing a polynucleotide that encodes an invention deacetylase and cells containing such vectors are also provided.

In another embodiment, the invention provides methods for identifying an inhibitor of acetyl glucosaminyl inositol deacetylase by contacting a candidate compound with an invention deacetylase in the presence of an acyl glucosaminyl inositol under suitable conditions and determining the presence or absence of breakdown products of the acyl glucosaminyl inositol indicative of deacetylase activity. Substantial absence of the breakdown product of deacetylase activity is indicative of a candidate compound that inhibits activity of the deacetylase.

In yet another embodiment, the present invention provides high throughput screening methods for identifying inhibitors of the invention deacetylases. The screening methods involve contacting each of a plurality of candidate compounds with an invention deacetylase in the presence of acyl glucosaminyl inositol under suitable conditions to form a plurality of reaction mixtures and determining the presence or absence of binding to the reaction mixtures of a detectable marker that binds to free amine. Substantial absence of binding of the marker to a reaction mixture in the invention screening methods is indicative of a candidate compound that inhibits activity of the deacetylase.

In yet another embodiment, the present invention provides derivatives of 1-D-myo-inosityl-2-amino-2-deoxy-α-D-glucopyranoside (GlcN-Ins), wherein the derivative contains a reactive residue attached to the amino group that promotes oxidative stress and wherein the derivative is selectively toxic to a mycothiol-producing actinomycete by being concentrated in the actinomycete.

In still another embodiment, the present invention provides live mutant actinomycetes. The invention mutant actinomycetes have a genome with a disruption in an endogenous acetyl glucosaminyl inositol deacetylase gene, wherein said disruption prevents function of an endogenous acetyl glucosaminyl inositol deacetylase while cell surface proteins and lipids are substantially unaffected, and wherein the disruption results in the mutant actinomycetes exhibiting survival in mammalian white blood cells for an immune response raising period of time.

In yet another embodiment, the present invention provides methods for decreasing the virulence of a pathogenic acetyl glucosaminyl inositol deacetylase-producing bacterium in mammalian cells by introducing into the bacterium an inhibitor of acetyl glucosaminyl inositol deacetylase activity. The intracellular presence of the inhibitor in the bacterium decreases activity of the deacetylase, thereby decreasing mycothiol biosynthesis by the bacterium as compared with untreated control bacterium. By virulence is meant the relative power and degree of pathogenicity possessed by organisms to produce disease as measured by clinical symptoms particular to the disease under consideration. For example, the virulence of a M. tuberculosis is measured with reference to the manifestation in an infected individual of the clinical symptoms recognized by a medical practitioner as indicative of tuberculosis.

In another embodiment, the present invention provides inhibitors of the invention acetyl glucosaminyl inositol deacetylases wherein the inhibitor is derived from GlcN-Ins by replacing the amino group therein with a moiety that chelates Zn²⁺, or otherwise binds the enzyme active site of the deacetylase.

In still another embodiment, the present invention provides methods for inhibiting growth of an acetyl glucosaminyl inositol-producing bacterium in a mammal, by administering to the mammal an effective amount of an inhibitor of intracellular acetyl glucosaminyl inositol deacetylase, thereby inhibiting growth of the bacterium in the mammal.

In yet another embodiment, the present invention provides processes for preparation of 1-D-myo-inosityl-2-amino-2-deoxy-α-D-glucopyranoside (GlcN-Ins) by contacting an acyl glucosaminyl inositol with an invention deacetylase under suitable conditions so as to hydrolyze the amide bond therein, and obtaining the GlcN-Ins.

### BRIEF DESCRIPTION OF THE DRAWINGS

FIGS. 1A and B show the nucleotide sequence of M. tuberculosis deacetylase gene mshB (Rv1170) (SEQ ID NO:7).

FIG. 1C shows the deduced amino acid sequence of M. tuberculosis GlcNAc-Ins deacetylase (SEQ ID NO:1) encoded by mshB.

FIG. 2 is a schematic drawing showing a map of plasmid pYA1170b employed to express Rv1170 in E. coli.

FIG. 3 is a chart showing the chemical structures of the substrates for assaying deacetylase and amidase activities of the invention acetyl glucosaminyl inositol deacetylases (MshB), such as MshB Rv1170 and homologs.

FIG. 4 is a graph showing growth of a mutant M. smegmatis (mutant 49) in 7H9 Middlebrook medium containing 17 μM GlcNAc-Ins. Mycothiol (MSH) and MSH precursor contents in cells are expressed in micromoles per gram of residual dry weight (RDW).

FIG. 5 is a schematic representation of the proposed MSH biosynthesis pathway.

FIG. 6 is a chart showing alignment of the amino acid sequences of four homologs of MshB of M. tuberculosis H37Rv (Rv1170) GenBank accession number B70875 and M. leprae GenBank accession number CAC30445. Sequences for M. avium (The Institute for Genomic Research (TIGR)), and M. smegmatis (Sanger Center) are from unfinished genomes.

FIG. 7 is a graph showing MshB deacetylase activity with 0.1 mM GlcNAc-Ins in the presence of 1,10-phenanthroline.

FIG. 8A is a graph showing MshB deacetylase reaction specific activity with 0.8 mM GlcNAc-Ins as substrate in the presence of increasing concentrations of thiol inhibitors.

FIG. 8B is a graph showing MshB amidase reaction specific activity with 0.55 mM MSmB as substrate in the presence of increasing concentrations of inhibitors.

FIG. 9 is a map of the plasmid pYA1170C used to express the recombinant His-tagged acetyl glucosaminyl inositol deacetylase encoded by M. tuberculosis Rv1170.

FIG. 10 is a graph showing percent survival of strains of M. smegmatis versus hydrogen peroxide concentration after treatment as described in Example 11 herein. Open triangles=mutant TN3, which produces no MSH; open circles=chemical mutant 164, which produces a low residual MSH content; and darkened squares=high MSH content mc² 155.

FIG. 11 is a graph showing percent survival over time of strains of M. smegmatis in murine macrophages as described in Example 11 herein. Open circles=chemical mutant TN3 (0% MSH content), Experiment 1; open triangles=mutant TN3 (0% MSH content), Experiment 2; and darkened squares=mc²155 (100% MSH content).

### DETAILED DESCRIPTION OF THE INVENTION

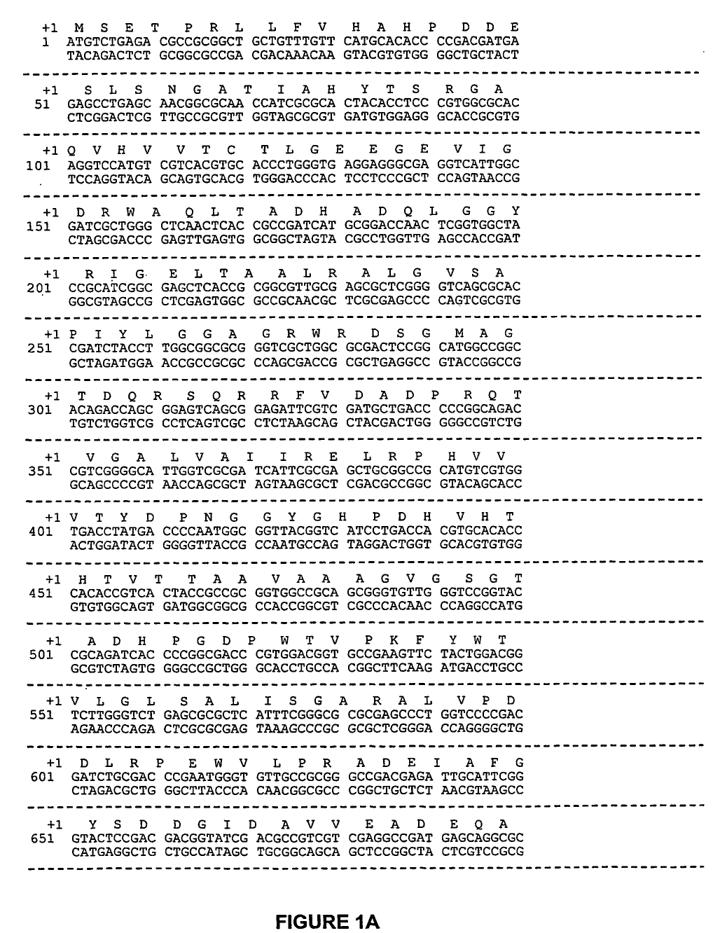

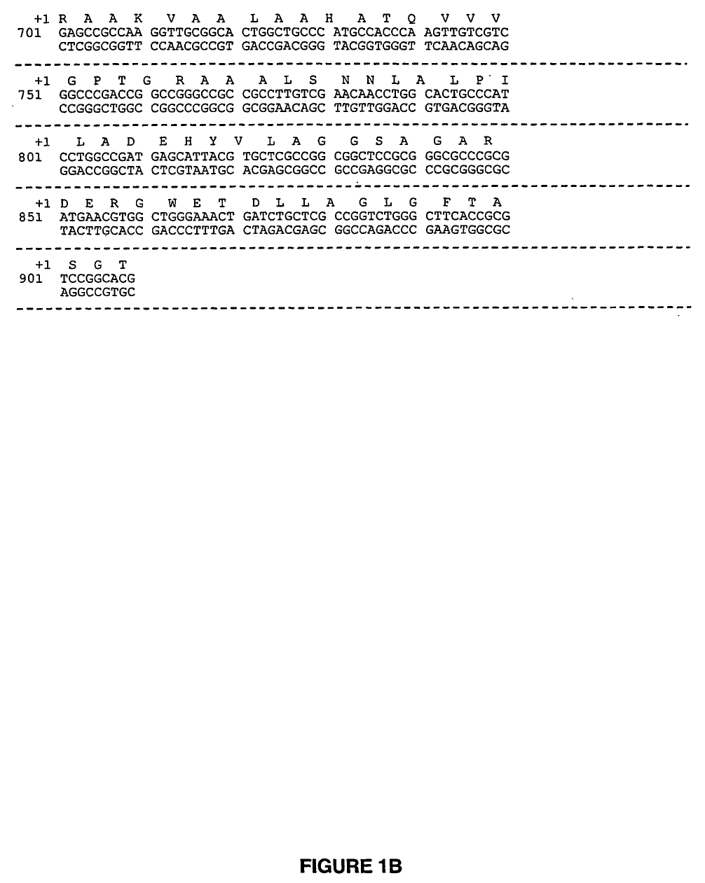

In accordance with the present invention, there are provided a family of purified acetyl glucosaminyl inositol deacetylase polypeptides with enzymatic deacetylase activity for acetyl glucosaminyl inositol. The invention acetyl glucosaminyl inositol deacetylases are characterized by having an N-terminal region with an amino acid sequence with 40% or greater sequence identity to an amino acid sequence as set forth in SEQ ID NO:2 and conservative variation thereof, four domains of conservation, wherein three of the domains contain conserved histidine residues, and deacetylase activity against acetyl glucosaminyl inositol. SEQ ID NO:2 corresponds to amino acid residues 1-100 of amino acids of SEQ ID NO:1 (FIG. 1C). The members of the family of invention deacetylases hydrolyze a C2-amide bond of acetyl glucosaminyl inositol moiety, such as N-acetyl-1-D-myo-inosityl-2-amino-2-deoxy-α-D-glucopyranoside (GlcNAc-Ins). Preferably the acetyl glucosaminyl inositol is a precursor of mycothiol (e.g., in a mycothiol producing bacterium).

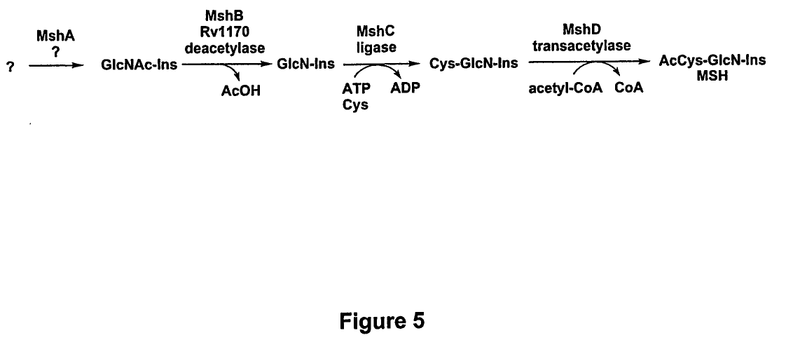

In preferred embodiments the three histidine-containing conserved domains in the invention deacetylases are selected from VHAHPDDE (SEQ ID NO:3), which corresponds to amino acids 10 through 17 of SEQ ID NO:1; VTCTLGEXGEV (SEQ ID NO:4), which corresponds to amino acids 38 through 49 of amino acid SEQ ID NO:1; YDPXGGYGHPDH (SEQ ID NO:5), which corresponds to amino acids 136 through 147 of SEQ ID NO:1; and AXXAHATQ (SEQ ID NO:6), which corresponds to amino acids 240 through 247 of amino acid SEQ ID NO:1 (FIG. 1C) wherein X is any amino acid; and conservative variations thereof, or any combination of any two or more thereof (See FIG. 6).

Members of the invention family of acetyl glucosaminyl inositol deacetylase polypeptides are metalloproteases. Accordingly, the invention further provides purified acetyl glucosaminyl inositol deacetylases that are characterized as having an N-terminal region with an amino acid sequence with at least 40% sequence identity to SEQ ID NO:2, one or more domains of conservation containing conserved metal chelating residues, and deacetylase activity against acetyl glucosaminyl inositol in the presence of metal ion. The invention deacetylases have been shown to contain an active site that contains a metal ion selected from the group consisting of Mn²⁺, Ni², Cd²⁺, Co²⁺, and Zn²⁺, with the preferred metal ion being Zn²⁺. The metal chelating residues in the invention deacetylases are preferably selected from the group consisting of histidine, aspartic acid and glutamic acid, and combinations thereof.

In preferred embodiments, the invention deacetylases have additional activity as amidases, catalyzing the hydrolysis of acyl glucosaminyl inositols. As used herein, the term “acyl glucosaminyl inositol” means substrate compounds for the invention family of deacetylases having the chemical formula R—CONH-GlcN-Ins wherein R═CH₃(CH₂)ₙ— where n=0-6. Included in the group of substrate compounds is the natural substrate, acetyl-GlcN-Ins, where n=0. Members of this group of substrate compounds are derived from an alkanoic acid. In addition, compounds having the chemical structure R—CONH-GlcN-Ins wherein R=aryl-(CH₂)ₙ, where n=0-6 are encompassed by the term. Members of this group of substrate compounds are derived from an aryl alkanoic acid such as phenyl-(CH₂)ₙCOOH.

The acyl glucosaminyl inositols of the above chemical formula can also be derived from the reaction of GlcN-Ins with any commercial acid chloride in the form RCOCl, wherein R is: o-tolyl-, 4-ethylphenyl-, 4-propylphenyl-, 4-biphenyl-, 3,4-dimethoxyphenyl-, 3,4,5-trimethoxyphenyl-, 2-furyl-, and the like.

The acyl glucosaminyl inositols of the above chemical formula can also be derived from an amino acid or an N-acetyl amino acid. Preferred amino acids include N-acetylcysteinyl-S—R′ or cysteinyl-S—R′, wherein R′ is an organic group attached to the cysteine sulfur, such as may be derived from commercial thiol labeling reagents like 2-bromoacetophenone, monobromobinane, N-ethylmaleimide, cerulenin, 7-diethylamino-3-(4′-maleimidyl phenyl)-4-methyl coumarin, 3(N-maleimidopropionyl)biocytin, and the like.

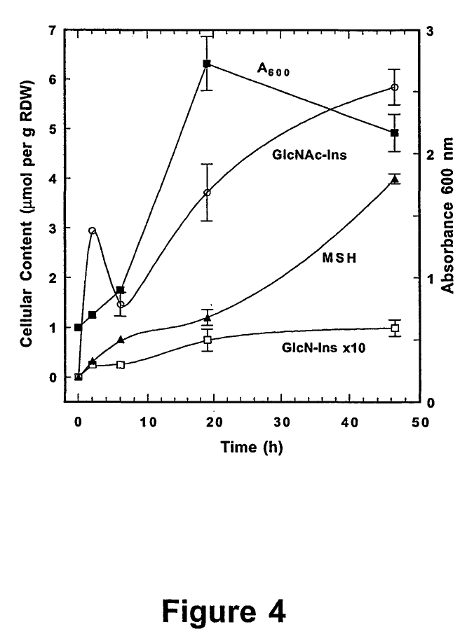

The studies described herein further elaborate the pathway involved in MSH biosynthesis. The present studies demonstrate that GlcNAc-Ins is a major intracellular MSH component in M. smegmatis and is converted to GlcN-Ins by GlcNAc-Ins deacetylase. This conversion defines the second step in MSH biosynthesis. The discovery described herein that Rv1170 is the gene encoding the deacetylase in the M. tuberculosis genome represents the first gene of the MSH biosynthesis pathway to be identified. Assuming that a single enzyme produces GlcNAc-Ins, it is proposed that the genes for the MSH biosynthesis pathway be designated mshA, mshB, mshC, and mshD, with the corresponding enzymes labeled as shown in FIG. 5. Identification of mshA, mshC, and mshD would be greatly simplified if they were clustered with mshB in a single operon, but inspection of the gene assignments and open reading frames surrounding mshB (Rv1170) in the M. tuberculosis genome indicates that this is not the case.

All mycobacteria thus far examined have high MSH content (G. L. Newton et al. (1996) J. Bacteriol. 178:1990-1995) and, based on the studies described herein, are expected to have an ortholog of the MSH biosynthesis enzyme MshB (Rv1170). The available unfinished mycobacterial genome databases yield homologs close to Rv1170 for Mycobacterium leprae (Sanger Centre), Mycobacterium bovis (Sanger Centre), M tuberculosis CDC1551 (The Institute for Genomic Research [TIGR]), M. smegmatis (TIGR), and Mycobacterium avium (TIGR). This sequence homology (FIG. 6) indicates that MSH biosynthesis in these organisms utilizes a GlcNAc-Ins deacetylase (MshB) in the same manner as that described here for M. smegmatis. Other actinomycetes that produce MSH are also expected to have a GlcNAc-Ins deacetylase (MshB) gene homologous to Rv1170.

Sequence BLAST searches to determine molecules with amino acid homology to the Rv1170 sequence of SEQ ID NO:1 (FIG. 1C) also show M. tuberculosis homolog Rv1082 (mycothiol S-conjugate deacetylase) and the Rv1082 orthologs from M. leprae, M. bovis (Sanger Centre), M. tuberculosis CDC1551 (TIGR), M. smegmatis (TIGR), and M. avium (TIGR) to be close homologs. Sequences with partial homology to Rv1170 were also found in the yeast, rat, and human genome databases. These genes code for PIG-L, the second enzyme in glycosylphosphatidylinositol (GPI) anchor biosynthesis (Nakamura et al., (1997) J. Biol. Chem. 272:15834-15840), and catalyze the deacetylation of N-acetylglucosaminyl-phosphatidylinositol (GlcNAc-PI).

The studies described in the Examples herein indicate that the invention deacetylase is rather specific for GlcNAc-Ins and is not a broad spectrum deacetylase. For example, removal of the inositol residue from the substrate GlcNAc-Ins resulted in a more than 300-fold decrease in reactivity (i.e., GlcNAc-Ins versus GlcNAc) (Table 1). Also, the studies described herein (Table 1) show that neither MSH nor MSmB is deacetylated by the invention deacetylases. Therefore, the AcCys moiety of MSH is inert to the deacetylase. This finding shows that the invention deacetylase has no significant activity for the degradation of MSH.

The invention deacetylase appears to be the control point for MSH biosynthesis. In cells tested, a very large endogenous pool of its substrate GlcNAc-Ins (12 to 15 μmol per g of residual dry weight (RDW)) was found (Table 2), but quite low levels were found of its product GlcN-Ins (˜0.2 μmol per g of RDW) (Table 2) and of the final intermediate Cys-GlcN-Ins (<0.006 μmol per g of RDW) leading to MSH. This finding shows that substantial quantities of MSH can be produced upon demand from the endogenous pool of GlcNAc-Ins under control of the invention family of acetyl glucosaminyl inositol deacetylases.

If a particular member of the invention family of polypeptide deacetylases hydrolyzes its substrate (GlcNAc-Ins), cleavage of the substrate molecule will form breakdown products wherein one product is a free amine (e.g., 1-D-myo-inosityl-2-amino-2-deoxy-α-D-glucopyranoside (GlcN-Ins)) and the other product is acetate. If a particular member of the invention family of polypeptide deacetylases has activity as an amide hydrolase (amidase), cleavage of the substrate acyl glucosaminyl inositol will form breakdown products wherein one product is a carboxylic acid, and the other product is a free amine (e.g., GlcN-Ins). If the substrate amide is a mycothiol-derived amide, one of the breakdown products will be GlcN-Ins and the other breakdown product will be a sulfur-containing carboxylic acid, such as an AcCys S-conjugate. AcCys S-conjugates are termed mercapturic acids, the final excreted product in the mercapturic acid pathway of glutathione-dependent detoxification in mammals.

Mycothiol (1-D-myo-inosityl-2-(N-acetylcysteinyl)amido-2-deoxy-α-D-glucopyranoside) (MSH) is present in a variety of actinomycetes and plays an essential role in a pathway of detoxification in such bacteria. Mycothiol is comprised of N-acetylcysteine (AcCys) amide linked to 1-D-myo-inosityl-2-amino-2-deoxy-α-D-glucopyranoside (GlcN-Ins) and is the major thiol produced by most actinomycetes. In the mycothiol-dependent detoxification process in actinomycetes, an alkylating agent is converted to a S-conjugate of mycothiol, the latter is cleaved to release a mercapturic acid, and the mercapturic acid is excreted from the cell (Newton et al. (2000) Biochemistry 39:10739-10746).

It has been discovered that invention acetyl glucosaminyl inositol deacetylases participate in the pathway of mycothiol biosynthesis in which substrate GlcNAc-Ins is an intermediate. In the mycothiol pathway as shown schematically in FIG. 5, GlcNAc-Ins is converted to GlcN-Ins by the invention deacetylase. In fact, the studies disclosed herein have shown that the invention deacetylases are the critical step in the production of mycothiol in actinomycetes. For example, it has been shown by the studies described herein (Example 10) that MshB is inhibited by mycothiol in a manner consistent with a negative feedback loop dependent upon the level of mycothiol for control of mycothiol biosynthesis in the cell. Thus, a falling level of mycothiol in the cell available for detoxification of intracellular toxins increases activity of the deacetylase so that substrate GlcNAc-Ins will be cleaved by the expressed deacetylase, in turn, providing the GlcN-Ins moiety necessary for mycothiol production.

A member of the invention family of polypeptide deacetylases shown to be responsible for cleavage of GlcNAc-Ins has been cloned from M. tuberculosis gene Rv1170 and has an amino acid sequence as set for in SEQ ID NO:1 (shown in FIG. 1C; see also GenBank Accession No. B70875) and a nucleotide sequence as set forth in SEQ ID NO:7 (shown in FIG. 1; see also nucleic acid residues 4222-5133 of GenBank Accession No. AL010186). The N-terminal region 100 residues of this newly discovered GlcNAc-Ins deacetylase has the amino acid sequence as shown in SEQ ID NO:2.

Members of invention family of acetyl glucosaminyl inositol deacetylases are formed in vivo by bacteria as part of a mycothiol biosynthesis pathway, most usually in bacteria characterized by intracellular production of mycothiol. Additional bacteria from which the invention deacetylase polypeptides can be derived include actinomycetes, such as M. smegmatis, M. leprae, M. bovis, M. intracellulare, M. africanum, M. marinarum, M. chelonai, Corynebacterium diphtheria, Actinomycetes israelii, M. avium complex (MAC) (Holzman, in Tuberculosis ed. by Rom and Gary (Little, Brown, and Company, 1996) Chapter 56), M. ulcerans, M. abscessus, or M. scrofulaceum, and the like. Homologous non-mycobacterial deacetylase proteins can also be derived from the antibiotic producers Streptomyces lincolnensis, Amycolatopsis mediterranei, Amycolatopsis orientalis, Streptomyces lavendulae, Streptomyces coelicolor, Streptomyces rochei, and the polyketide erythromycin antibiotic producer Saccharopolyspora erythraea.

Inhibitors of the invention acetyl glucosaminyl inositol deacetylases are particularly well suited as antibiotics since mycothiol production will cease in the absence of the product GlcN-Ins produced by activity of the invention deacetylases. Accordingly, in another embodiment according to the present invention, there are provided methods for identifying an inhibitor of an invention acetyl glucosaminyl inositol deacetylase, MshB. The invention methods comprise contacting a candidate compound with a member of the invention family of deacetylases in the presence of a substrate for the enzyme under suitable conditions and determining the presence or absence of breakdown products of the substrate indicative of deacetylase (or optionally S-conjugate amidase) activity. For example, if the test compound is a putative inhibitor of amide hydrolase activity of the invention polypeptide deacetylase, the absence of acetate or other carboxylic acid and/or free amine, such as GlcN-Ins, as breakdown products indicates the candidate compound is an inhibitor of the activity of the invention polypeptide as a deacetylase. The preferred substrate for testing for deacetylase activity of the invention polypeptides is the acetyl glucosaminyl inositol (GlcNAc-Ins). On the other hand, if the test compound is a putative inhibitor of MshB, the appearance of free amine, such as GlcN-Ins, and/or acetate or carboxylate indicates that the candidate compound is not an inhibitor of the activity of the invention deacetylase as a deacetylase or as an amidase of an acyl glucosaminyl inositol. A preferred substrate for use in screening for inhibitors of deacetylase activity is N-acetyl-1-D-myo-inosityl-2-amino-2-deoxy-α-D-glucopyranoside (GlcNAc-Ins).

Substantial absence of the breakdown products of enzymatic activity is indicative of a candidate compound that inhibits enzymatic activity of the deacetylase. In a preferred embodiment, the deacetylase used in the invention method is an acetyl glucosaminyl inositol deacetylase and the method is designed to assay for production of free amine. Such assays can be conducted en masse using a high throughput screening method for identifying inhibitors of the invention deacetylase. For example, a plurality of candidate compounds can be contacted with an invention deacetylase in the presence of a substrate for the enzyme (e.g. an acyl glucosaminyl inositol) under suitable conditions (e.g., as shown in the Examples herein) for activity of the deacetylase. Binding of a marker selected to bind to a breakdown product can then be assayed using any technique known in the art for high throughput screening. For example, a colorometric or fluorometric marker that binds to free amine can be used to screen a plurality of reaction mixtures formed in the wells of a microtitre plate (Wong, et al. (2001) Anal. Biochem. 290:338-346). A preferred fluorometric marker for use in the invention high throughput screening methods is fluorescamine, which binds to free amine.

In another embodiment of the invention, methods are provided for determination of acetyl glucosaminyl inositol (GlcNAc-Ins) in a sample by contacting a sample containing GlcNAc-Ins with an invention deacetylase under suitable conditions and determining the amount of GlcN-Ins produced, wherein the amount of GlcN-Ins produced is a measure of the GlcNAc-Ins in the sample. The amount of GlcN-Ins in the sample can be determined by HPLC after labeling thereof with a fluorometric or colorimetric reagent, such as 6-aminoquinolyl-N-hydroxysuccinimidyl carbamate (sold as AccQFluor® reagent by Waters), and the like.

In an alternative embodiment of the invention, methods are provided for decreasing the virulence in mammalian cells of a pathogenic acetyl glucosaminyl inositol deacetylase-producing bacterium, such as an actinomycete. In the invention method for decreasing the virulence of pathogenic acetyl glucosaminyl inositol deacetylase-producing bacteria, an inhibitor of acetyl glucosaminyl inositol deacetylase (for example, one identified by the above-described screening method) is introduced into the bacterium. Intracellular uptake by the treated bacterium of the inhibitor results in decreased activity of the deacetylase, thereby decreasing mycothiol biosynthesis by the bacterium as compared with untreated control bacterium. For example, for treatment of isolated mammalian cells, the introducing can comprise culturing the bacterium in the presence of the inhibitor. Alternatively, for treatment of mammalian cells contained in a living organism, the inhibitor may be administered systemically to the living organism. Pathogenic acetyl glucosaminyl inositol deacetylase-producing bacteria whose virulence can be reduced according to the invention methods include such actinomycetes as M. smegmatis, M. tuberculosis, M. leprae, M. bovis particularly in bovine subjects), M. intracellulare, M. africanum, and M. marinarum. M. chelonai, Corynebacterium diphtheriae, Actinomyces israelii, M. avium complex (MAC), M. ulcerans, M. abscessus, M. scrofulaceum, and the like.

The inhibitors used in the invention methods for decreasing the virulence of a pathogenic acetyl glucosaminyl inositol deacetylase-producing bacterium may either inhibit intracellular production of the deacetylase or inhibit intracellular deacetylase activity of the deacetylase. In one embodiment according to the present invention, there are provided inhibitors of the members of the invention family of deacetylase polypeptides. The invention inhibitors are characterized by being derived from GlcN-Ins by replacing the amino group therein with a moiety that chelates Zn²⁺ or otherwise binds the enzyme active site. The invention inhibitors may also chelate one or more metal ions selected from the group consisting of Mn²⁺ and Ni², Cd²⁺, and Co²⁺. The following are examples of moieties that can substitute for the amino group in GlcN-Ins in the invention inhibitors:
ClCH₂CONH—
HONHCONH—
HONHCOCH₂—
HOPO(CH₃)NH—
HOPO(CH₃)ₙCH₂—; wherein n=1-5
HSCH₂(CH₂)ₙNH—; wherein n=2-5
HS(CH₂)ₙCONH—; wherein n=1-3.

In another embodiment according to the present invention, there are provided derivatives of GlcN-Ins, wherein the derivative contains a reactive residue attached to the amino group that promotes oxidative stress (e.g., including nitrosative stress) so as to be selectively toxic to a mycothiol-producing actinomycete by being concentrated in the actinomycete. Examples of reactive residues that can be attached to the amino group of GlcN-Ins to cause the compound to be selectively toxic to mycothiol-producing actinomycetes by intracellular concentration include, but are not limited to a nitroso residue, a nitroalkyl residue comprising 1 to 3 carbon atoms, a S-nitrosomercaptoalkyl residue comprising 2 to 4 carbon atoms, or a peroxyalkyl residue comprising 2 to 4 carbon atoms.

In another embodiment according to the present invention, the invention inhibitor is an anti-sense oligonucleotide complementary to a target region in a messenger RNA that encodes a polypeptide having an amino acid sequence segment with 40% or more sequence identity to the amino acid sequence of SEQ ID NO:2 or 80% or more sequence identity to at least one amino acid sequence selected from the group consisting of SEQ ID NOS: 3, 4, 5, 6 and conservative variations thereof. In another embodiment the anti-sense oligonucleotide hybridizes under intracellular conditions with a messenger RNA that encodes a polypeptide having an N-terminal amino acid sequence as set forth in SEQ ID NO:2.

For example, in one embodiment, the candidate compound inhibits intracellular production or activity of the acetyl glucosaminyl inositol deacetylase. A presently preferred drug candidate for screening in live bacteria for activity that inhibits intracellular production or activity of acetyl glucosaminyl inositol deacetylase is an anti-sense oligonucleotide complementary to a target region in a messenger RNA that encodes a polypeptide having an N-terminal amino acid sequence with 40% or more sequence identity to the amino acid sequence set forth in SEQ ID NO:2, or a conservative variation thereof, for example, 50%, 55%, 60% or 65% sequence identity. Suitable conditions for conducting invention drug screening methods are well known in the art and are described, for example, in the Examples hereinbelow.

Alternatively, an anti-sense oligonucleotide can be designed to hybridize under in vivo conditions with a messenger RNA that encodes a polypeptide having an N-terminal amino acid sequence as set forth in SEQ ID NO:2, or contains an amino acid segment as set forth in SEQ ID NOs:3, 4, 5, and 6, or a conservative variation thereof.

The anti-sense oligonucleotide can comprise from about 10 to about 60 nucleic acid residues, for example from 10 to about 50, or from 10 to about 40, 30 or 20 nucleic acid residues. “Hybridization” refers to the process by which a nucleic acid strand joins with a complementary strand through base pairing. Hybridization reactions can be sensitive and selective so that a particular sequence of interest will join with a complementary strand even in samples in which it is present at low concentrations. Suitable intracellular conditions for hybridization of an anti-sense oligonucleotide to messenger RNA will be determined by the particular bacterium used in the invention method. In general, the pH, temperature and salt concentration must be comparable to intracellular conditions in the test bacterium.

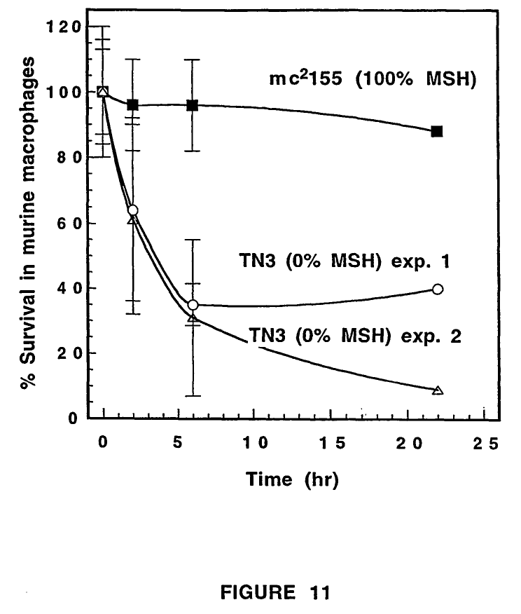

In yet another embodiment according to the present invention, there are provided live mutant actinomycetes, whose genomes comprise a disruption in an endogenous acetyl glucosaminyl inositol deacetylase gene and thereby prevents mycothiol synthesis. Disruption of genes for mycothiol biosynthesis in mycobacteria reduces their survival in mammalian macrophages (FIG. 11). Disruption of the endogenous acetyl glucosaminyl inositol deacetylase gene will prevent function of an endogenous acetyl glucosaminyl inositol deacetylase while cell surface proteins and lipids should be substantially unaffected. As a result, invention live mutant actinomycetes exhibit the phenotype of transient survival in mammalian white blood cells, such as murine or human white blood cells, for an immune response raising period of time. Such genetically engineered live mutant actinomycetes will survive in mammalian white blood cells for a period of time from 1 to 30 days, for example from 4 to 25 days or from 5 to 20 days, but in no event for more than 30 days. Due to lack of intracellular acetyl glucosaminyl inositol deacetylase the invention live mutant bacterium will fail to produce mycothiol. Hence, the mutant live bacterium is unable to survive the oxidative stress inherent in the intracellular environment of mammalian white blood cells long enough to establish infection in the cells or to establish infection in an immunocompetent mammal containing such white blood cells.

Thus, invention live mutant actinomycetes possess a combination of features desired for a vaccine effective in mammals against infection by such pathogenic actinomycetes as M. smegmatis, M. tuberculosis, M. leprae, M. bovis, M. intracellulare, M. africanum, M. marinarum, M. chelonai, Corynebacterium diphtheria, Actinomycetes israelii, M. avium complex (MAC), M. ulcerans, M. abscessus, M. scrofulaceum, and the like. An individual (e.g., an animal, such as a mouse, a farm animal or a human) to which the live mutant is administered according to a protocol appropriate for inducing a protective immune response and who has not previously been infected by the counterpart wild type will mount an immune response to the vaccine, for example an immune response sufficient to protect the individual against future infection by the corresponding wild-type live pathogen. Alternatively, the invention live mutant actinomycetes are useful as a research tool to investigate the properties desirable in a live mutant vaccine.

In still another embodiment according to the present invention, there are provided processes for preparation of GlcN-Ins by contacting an N-acyl glucosaminyl inositol under suitable conditions with an acetyl glucosaminyl inositol deacetylase so as to hydrolyze the amide bond therein to obtain stereochemically pure α(1-1) GlcN-Ins (1-D-myo-inosityl-2-amino-2-deoxy-α-D-glucopyranoside). For example, the N-acyl glucosaminyl inositol can be a mycothiol S-conjugate, such as the bimane derivative of mycothiol. The deacetylase cleaves the N-acyl glucosaminyl inositol, freeing GlcN-Ins as one of the cleavage breakdown products. GlcN-Ins has utility in conducting research regarding deacetylase activity and mycothiol biochemistry in bacteria, development of products and procedures for overcoming the antibiotic resistance of pathogenic bacteria, such as actinomycetes, and as a precursor for formation of acyl glucosaminyl inositol derivatives and inhibitors of deacetylases thereof as disclosed herein.

A “conservative variation” in an amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from an deacetylase polypeptide, resulting in modification of the structure of the polypeptide, without significantly altering its biological activity. For example, carboxyl-terminal amino acids that are not required for deacetylase or deacetylase activity of the invention polypeptides can be removed.

Biologically functional viral and plasmid DNA vectors capable of expression and replication in a host are known in the art. Such vectors are used to incorporate DNA sequences of the invention. In general, expression vectors containing promoter sequences which facilitate the efficient transcription of the inserted eukaryotic genetic sequence are used in connection with the host. The expression vector typically contains an origin of replication, a promoter, and a terminator, as well as specific genes that are capable of providing phenotypic selection of the transformed cells.

In addition to expression vectors known in the art such as bacterial, yeast and mammalian expression systems, baculovirus vectors may also be used. One advantage to expression of foreign genes in this invertebrate virus expression vector is that it is capable of expression of high levels of recombinant proteins, which are antigenically and functionally similar to their natural counterparts. Baculovirus vectors and the appropriate insect host cells used in conjunction with the vectors will be known to those skilled in the art.

The term “recombinant expression vector” refers to a plasmid, virus or other vehicle known in the art that has been manipulated by insertion or incorporation of the invention acetyl glucosaminyl inositol deacetylase genetic sequences. Such expression vectors contain a promoter sequence that facilitates the efficient transcription of the inserted genetic sequence of the host. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg, et al., Gene, 56:125, 1987), the pMSXND expression vector for expression in mammalian cells (Lee and Nathans, J. Biol. Chem., 263:3521, 1988) and baculovirus-derived vectors for expression in insect cells. The DNA segment can be present in the vector operably linked to regulatory elements, for example, a promoter (e.g., T7, metallothionein I, or polyhedrin promoters).

The vector may include a phenotypically selectable marker to identify host cells which contain the expression vector. Examples of markers typically used in prokaryotic expression vectors include antibiotic resistance genes for ampicillin (β-lactamases), tetracycline and chloramphenicol (chloramphenicol acetyltransferase). Examples of such markers typically used in mammalian expression vectors include the gene for adenosine deaminase (ADA), aminoglycoside phosphotransferase (neo, G418), dihydrofolate reductase (DHFR), hygromycin-B-phosphotransferase (HPH), thymidine kinase (TK), and xanthine guanine phosphoribosyltransferse (XGPRT, gpt).

The isolation and purification of host cell expressed polypeptides of the invention may be by any conventional means such as, for example, preparative chromatographic separations and immunological separations such as those involving the use of monoclonal or polyclonal antibody.

Transformation of the host cell with the recombinant DNA may be carried out by conventional techniques well known to those skilled in the art. Where the host is prokaryotic, such as E. coli, competent cells which are capable of DNA uptake can be prepared from cells harvested after exponential growth and subsequently treated by electroporation or the CaCl₂ method using procedures well known in the art. Alternatively, MgCl₂ or RbCl could be used.

Where the host used is a eukaryote, various methods of DNA transfer can be used. These include transfection of DNA by calcium phosphate-precipitates, conventional mechanical procedures such as microinjection, insertion of a plasmid encased in liposomes, or the use of virus vectors. Eukaryotic cells can also be cotransformed with DNA sequences encoding the polypeptides of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (Eukaryotic Viral Vectors, Cold Spring Harbor Laboratory, Gluzman ed., 1982). Examples of mammalian host cells include COS, BHK, 293, and CHO cells.

Eukaryotic host cells may also include yeast. For example, DNA can be expressed in yeast by inserting the DNA into appropriate expression vectors and introducing the product into the host cells. Various shuttle vectors for the expression of foreign genes in yeast have been reported (Heinemann, J. et al., Nature, 340:205, 1989; Rose, M. et al., Gene, 60:237, 1987).

The invention provides antibodies that are specifically reactive with invention deacetylase polypeptides or fragments thereof.

Antibody that consists essentially of pooled monoclonal antibodies with different epitopic specificities, as well as distinct monoclonal antibody preparations are provided. Monoclonal antibodies are made from antigen containing fragments of the protein by methods well known in the art (Kohler, et al., Nature, 256:495, 1975; Current Protocols in Molecular Biology, Ausubel, et al., ed., 1989). Monoclonal antibodies specific for acetyl glucosaminyl inositol deacetylase polypeptide can be selected, for example, by screening for hybridoma culture supernatants that react with acetyl glucosaminyl inositol deacetylase polypeptides, but do not react with other bacterial deacetylases.

Antibody that consists essentially of pooled monoclonal antibodies with different epitopic specificities, as well as distinct monoclonal antibody preparations are provided. Monoclonal antibodies are made from antigen containing fragments of the protein by methods well known in the art (Kohler, et al., Nature, 256:495, 1975; Current Protocols in Molecular Biology, Ausubel, et al., ed., 1989).

The term “antibody” as used in this invention includes intact molecules as well as fragments thereof, such as Fab, F(ab′)₂ and Fv, which are capable of binding the epitopic determinant. These antibody fragments retain some ability to selectively bind with its antigen or receptor and are defined as follows:
(1) Fab, the fragment which contains a monovalent antigen-binding fragment of an antibody molecule can be produced by digestion of whole antibody with the enzyme papain to yield an intact light chain and a portion of one heavy chain;
(2) Fab′, the fragment of an antibody molecule can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)₂ is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York (1988), incorporated herein by reference).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

Antibodies that bind to acetyl glucosaminyl inositol deacetylase polypeptide of the invention can be prepared using an intact polypeptide or fragments containing small peptides of interest as the immunizing antigen. The polypeptide or a peptide used to immunize an animal can be derived from translated cDNA or chemical synthesis and can be conjugated to a carrier protein, if desired. Such commonly used carriers, which are chemically coupled to the peptide, include keyhole limpet hemocyanin (KLH), thyroglobulin, bovine serum albumin (BSA), and tetanus toxoid. The coupled peptide is then used to immunize the animal (e.g., a mouse, a rat, or a rabbit).

If desired, polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1994, incorporated herein by reference).

It is also possible to use the anti-idiotype technology to produce monoclonal antibodies that mimic an epitope. For example, an anti-idiotypic monoclonal antibody made to a first monoclonal antibody will have a binding domain in the hypervariable region that is the “image” of the epitope bound by the first monoclonal antibody.

In yet other preferred embodiments, the recombinant acetyl glucosaminyl inositol deacetylase polypeptide is a fusion protein further-comprising a second polypeptide portion having an amino acid sequence from a protein unrelated to the acetyl glucosaminyl inositol deacetylase. Such fusion proteins can be functional in a two-hybrid assay.

Another aspect of the present invention provides a substantially pure nucleic acid having a nucleotide sequence which encodes an acetyl glucosaminyl inositol deacetylase polypeptide, or a fragment thereof, having an amino acid sequence at least 60% homologous to one of SEQ ID NOs:2, 3, 4 or 5. In a more preferred embodiment, the nucleic acid encodes a protein having an amino acid sequence 40% or more homologous to SEQ ID NO:2, more preferably at least 50% homologous to SEQ ID NO:2, and most preferably at least 65% homologous to SEQ ID NO:2.

In another embodiment, the nucleic acid hybridizes under stringent conditions to a nucleic acid probe corresponding to at least 12 consecutive nucleotides encoding SEQ ID NO:3; more preferably to at least 20 consecutive nucleotides encoding SEQ ID NO:3; more preferably to at least 24 consecutive nucleotides encoding SEQ ID NO:3.

In a further embodiment, the nucleic acid hybridizes under stringent conditions to a nucleic acid probe corresponding to at least 12 consecutive nucleotides encoding SEQ ID NO:4; more preferably to at least 20 consecutive nucleotides encoding SEQ ID NO:4; more preferably to at least 30 consecutive nucleotides encoding SEQ ID NO:4.

In yet a further embodiment, the nucleic acid hybridizes under stringent conditions to a nucleic acid probe corresponding to at least 12 consecutive nucleotides encoding SEQ ID NO:5; more preferably to at least 20 consecutive nucleotides encoding SEQ ID NO:5; more preferably to at least 30 consecutive nucleotides encoding SEQ ID NO:5.

In yet a further embodiment, the nucleic acid hybridizes under stringent conditions to a nucleic acid probe corresponding to at least 12 consecutive nucleotides encoding SEQ ID NO:6; more preferably to at least 20 consecutive nucleotides encoding SEQ ID NO:6.

Furthermore, in certain embodiments, the acetyl glucosaminyl inositol deacetylase nucleic acid will comprise a transcriptional regulatory sequence, e.g. at least one of a transcriptional promoter or transcriptional enhancer sequence, operably linked to the acetyl glucosaminyl inositol deacetylase-gene sequence so as to render the recombinant acetyl glucosaminyl inositol deacetylase gene sequence suitable for use as an expression vector.

The present invention also features transgenic non-human organisms, e.g. live mutant actinomycetes, which either express a heterologous acetyl glucosaminyl inositol deacetylase gene, or in which expression of their own acetyl glucosaminyl inositol deacetylase gene is disrupted. In addition to the other utilities of such organisms disclosed herein, such a transgenic organism has utility for overproduction of acetyl glucosaminyl inositol needed for screening (particularly high throughput screening) for compounds that inhibit acetyl glucosaminyl inositol deacetylase activity in mycothiol-producing bacteria.

The present invention also provides a probe/primer comprising a substantially purified oligonucleotide, wherein the oligonucleotide comprises a region of nucleotide sequence which hybridizes under stringent conditions to at least 10 consecutive nucleotides of sense or anti-sense sequence encoding one of the amino acid sequences encompassed by SEQ ID NOs:2, 3, 4 5, or 6, or naturally occurring mutants thereof.

Yet another aspect of the invention pertains to a peptidomimetic that binds to or interferes with an acetyl glucosaminyl inositol deacetylase polypeptide and inhibits its binding to or cleavage of substrate acyl glucosaminyl inositols. For example, a preferred peptidomimetic is an analog of a peptide having the sequence of one of the SEQ ID NOs. 1, 2, 3, 4, 5, or 6. Non-hydrolyzable peptide analogs of such residues can be generated using, for example, benzodiazepine, azepine, substituted gama-lactam rings, keto-methylene pseudopeptides, beta-turn dipeptide cores, or beta-aminoalcohols.

Other features and advantages of the invention will be apparent from the detailed description herein, and from the claims. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

As used herein, the term “actinomycetes” and “an actinomycete” encompasses any bacterium of the order Actinomycetales.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or anti-sense) and double-stranded polynucleotides.

As used herein, the terms “gene”, “recombinant gene” and “gene construct” refer to a nucleic acid comprising an open reading frame encoding an invention acetyl glucosaminyl inositol deacetylase, including both exon and (optionally) intron sequences. The term “intron” refers to a DNA sequence present in a given acetyl glucosaminyl inositol deacetylase gene which is not translated into protein and is generally found between exons.

“Homology” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. Percent homology between sequences is a function of the number of matching or homologous positions shared by the sequences.

The sequence data of a test clone is aligned to the sequences in the database or databases using algorithms designed to measure homology between two or more sequences. Sequence alignment methods include, for example, BLAST (Altschul et al., 1990), BLITZ (MPsrch) (Sturrock & Collins, 1993), and FASTA (Person & Lipman, 1988). For example, optimal alignment of sequences for aligning a comparison window may be conducted by the local homology algorithm of Smith (Smith and Waterman, Adv Appl Math, 1981; Smith and Waterman, J Teor Biol, 1981; Smith and Waterman, J Mol Biol, 1981; Smith et al, J Mol Evol, 1981), by the homology alignment algorithm of Needleman (Needleman and Wuncsch, 1970), by the search of similarity method of Pearson (Pearson and Lipman, 1988), by computerized implementations of these algorithms (GAP, BESTFIT, FASTA, and TFASTA in the Wisconsin Genetics Software Package Release 7.0, Genetics Computer Group, 575 Science Dr., Madison, Wis., or the Sequence Analysis Software Package of the Genetics Computer Group, University of Wisconsin, Madison, Wis.), or by inspection, and the best alignment (i.e., resulting in the highest percentage of homology over the comparison window) generated by the various methods is selected.

The term “transfection” or “transforming” and grammatical equivalents thereof, refers to the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of one of the invention family of acetyl glucosaminyl inositol deacetylases.

“Cells” or “cell cultures” or “recombinant host cells” or “host cells” are often used interchangeably as will be clear from the context. These terms include the immediate subject cells that express the acetyl glucosaminyl inositol deacetylases of the present invention, and, of course, the progeny thereof. It is understood that not all progeny are exactly identical to the parental cell, due to chance mutations or difference in environment. However, such altered progeny are included in these terms, so long as the progeny retain the characteristics relevant to those conferred on the originally transformed cell. In the present case, such a characteristic might be the ability to produce a recombinant acetyl glucosaminyl inositol deacetylase polypeptide.

As used herein, the term “vector” refers to a nucleic acid molecule capable of transporting another nucleic acid to which it has been linked. The term “expression vector” includes plasmids, cosmids or phages capable of synthesizing the subject acetyl glucosaminyl inositol deacetylase polypeptide encoded by the respective recombinant gene carried by the vector. Preferred vectors are those capable of autonomous replication and/expression of nucleic acids to which they are linked. In the present specification, “plasmid” and “vector” are used interchangeably as the plasmid is the most commonly used form of vector. Moreover, the invention is intended to include such other forms of expression vectors which serve equivalent functions and which become known in the art subsequently hereto.

“Transcriptional regulatory sequence” is a generic term used throughout the specification to refer to DNA sequences, such as initiation signals, enhancers, and promoters, as well as polyadenylation sites, which induce or control transcription of protein coding sequences with which they are operably linked. In preferred embodiments, transcription of a recombinant acetyl glucosaminyl inositol deacetylase gene is under the control of a promoter sequence (or other transcriptional regulatory sequence) that controls the expression of the recombinant gene in a cell-type in which expression is intended. It will also be understood that the recombinant gene can be under the control of transcriptional regulatory sequences which are the same or which are different from those sequences that control transcription of the naturally occurring form of the regulatory protein.

As used herein, a “transgenic organism” is any organism, preferably a bacteria in which one or more of the cells of the organism contain heterologous nucleic acid introduced by way of human intervention, such as by transgenic techniques well known in the art. The nucleic acid is introduced into the cell by way of deliberate genetic manipulation, such as by microinjection or by infection with a recombinant virus or a vector. The term genetic manipulation does not include classical crossbreeding, or in vitro fertilization, but rather is directed to the introduction of a recombinant DNA molecule. This molecule may be integrated within a chromosome, or it may be extrachromosomally replicating DNA. In the typical transgenic organisms described herein, the transgene causes cells to express a recombinant form of the subject acetyl glucosaminyl inositol deacetylase polypeptides.

As used herein, the term “transgene” means a nucleic acid sequence (encoding, e.g., an acetyl glucosaminyl inositol deacetylase polypeptide), which is partly or entirely heterologous, i.e., foreign, to the transgenic organism or cell into which it is introduced, or, is homologous to an endogenous gene of the transgenic organism or cell into which it is introduced, but which is designed to be inserted, or is inserted, into the organism's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

The term “evolutionarily related to”, with respect to nucleic acid sequences encoding acetyl glucosaminyl inositol deacetylase polypeptides, refers to nucleic acid sequences that have arisen naturally in an organism, including naturally occurring mutants. The term also refers to nucleic acid sequences which, while derived from a naturally occurring acetyl glucosaminyl inositol deacetylase polypeptide, have been altered by mutagenesis, as for example, combinatorial mutagenesis, yet still encode polypeptides which have the deacetylase activity of an acetyl glucosaminyl inositol deacetylase polypeptide.

One aspect of the present invention pertains to an isolated nucleic acid comprising the nucleotide sequence encoding an acetyl glucosaminyl inositol deacetylase polypeptide, fragments thereof encoding polypeptides having acetyl glucosaminyl inositol deacetylase activity, and/or equivalents of such nucleic acids. The term-nucleic acid as used herein is intended to include such fragments and equivalents. The term equivalent is understood to include nucleotide sequences encoding functionally equivalent acetyl glucosaminyl inositol deacetylase polypeptides or functionally equivalent peptides having an activity of an acetyl glucosaminyl inositol deacetylase polypeptide such as described herein. Equivalent nucleotide sequences will include sequences that differ by one or more nucleotide substitutions, additions or deletions, such as allelic variants; and will also include sequences that differ from the nucleotide sequence encoding native acetyl glucosaminyl inositol deacetylases due to the degeneracy of the genetic code. Equivalents will also include nucleotide sequences that hybridize under stringent conditions (i.e., equivalent to about 20-27° C. below the melting temperature of the DNA duplex formed in about 1 M salt) to the nucleotide sequence of an acetyl glucosaminyl inositol deacetylase gene, such as that as set forth in SEQ ID NO:7, particularly those segments encoding the polypeptides shown in one of SEQ ID NOs. 2, 3, 4, 5 or 6, and conservative variations thereof. In one embodiment, equivalents will further include nucleic acid sequences derived from and evolutionarily related to such nucleotide sequences.

The term “isolated” or “purified” as also used herein with respect to nucleic acids, such as DNA or RNA, refers to molecules separated from other DNAs, or RNAs, respectively, that are present in the natural source of the macromolecule. For example, an isolated nucleic acid encoding one of the subject acetyl glucosaminyl inositol deacetylase polypeptides preferably includes no more than 10 kilobases (kb) of nucleic acid sequence which naturally immediately flanks the acetyl glucosaminyl inositol deacetylase gene in genomic DNA, more preferably no more than 5 kb of such naturally occurring flanking sequences, and most preferably less than 1.5 kb of such naturally occurring flanking sequence. The term isolated or purified as used herein also refers to a nucleic acid or peptide that is substantially free of cellular material or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state.

In yet another embodiment, the nucleic acid of the invention encodes a peptide having an amino acid sequence as shown SEQ ID NO:1. Preferred nucleic acids encode a peptide having acetyl glucosaminyl inositol deacetylase polypeptide activity and being 40% or more homologous, more preferably 50% homologous and most preferably 65% homologous with an amino acid sequence as set forth in SEQ ID NO:2 (encoded by a nucleic acid sequence as set forth in SEQ ID NO:7). Nucleic acids that encode peptides having an activity of an invention acetyl glucosaminyl inositol deacetylase polypeptide are also within the scope of the invention.

Another aspect of the invention provides a nucleic acid which hybridizes under high or low stringency conditions to a nucleic acid which encodes an acetyl glucosaminyl inositol deacetylase polypeptide having all or a portion of an amino acid sequence shown in one of SEQ ID NOs. 2, 3, 4, 5 or 6. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C.

Isolated nucleic acids that differ from the nucleotide sequences disclosed herein due to degeneracy in the genetic code are also within the scope of the invention. For example, more than one triplet designates a number of amino acids. Codons that specify the same amino acid, or synonyms (for example, CAU and CAC are synonyms for histidine) may result in “silent” mutations that do not affect the amino acid sequence of the protein. However, it is expected that DNA sequence polymorphisms that do lead to changes in the amino acid sequences of the subject acetyl glucosaminyl inositol deacetylase polypeptides will exist among prokaryotic cells. One skilled in the art will appreciate that these variations in one or more nucleotides (up to about 3-4% of the nucleotides) of the nucleic acids encoding a particular member of the acetyl glucosaminyl inositol deacetylase polypeptide family may exist among individuals of a given species due to natural allelic variation. Any and all such nucleotide variations and resulting amino acid polymorphisms are within the scope of this invention.

Fragments of the nucleic acid encoding a biologically active portion of the subject acetyl glucosaminyl inositol deacetylase polypeptides are also within the scope of the invention. As used herein, a fragment of the nucleic acid encoding an active portion of an acetyl glucosaminyl inositol deacetylase polypeptide refers to a nucleotide sequence having fewer nucleotides than the nucleotide sequence encoding the full length amino acid sequence of, for example, the deacetylase polypeptides represented in nucleic acid SEQ ID NO:1, and which encodes a peptide which retains at least a portion of the biological activity of the full-length protein (i.e., a peptide capable of acetyl glucosaminyl inositol deacetylase activity) as defined herein, or alternatively, which is functional as an antagonist of the deacetylase activity of the full-length protein. Nucleic acid fragments within the scope of the invention include those capable of hybridizing under high or low stringency conditions with nucleic acids from other species, e.g. for use in screening protocols to detect homologs of the subject acetyl glucosaminyl inositol deacetylase polypeptides. Nucleic acids within the scope of the invention may also contain linker sequences, modified restriction endonuclease sites and other sequences useful for molecular cloning, expression or purification of such recombinant peptides.

As indicated by the examples set out below, a nucleic acid encoding a peptide having an activity of an invention deacetylase polypeptide may be obtained from mRNA or genomic DNA present in any of a number of antibiotic-producing or pathogenic bacteria, particularly actinomycetes, in accordance with protocols described herein, as well as those generally known to those skilled in the art. A cDNA encoding an acetyl glucosaminyl inositol deacetylase polypeptide, for example, can be obtained by isolating total mRNA from a bacterial cell. Double stranded cDNAs can then be prepared from the total mRNA, and subsequently inserted into a suitable plasmid or bacteriophage vector using any one of a number of known techniques. A gene encoding an acetyl glucosaminyl inositol deacetylase polypeptide can also be cloned using established polymerase chain reaction techniques in accordance with the nucleotide sequence information provided by the invention.

Another aspect of the invention relates to the use of an “anti-sense” isolated nucleic acid. As used herein, an “anti-sense” inhibition of endogenous production of an acetyl glucosaminyl inositol deacetylase molecule is carried out by administration or in situ generation of oligonucleotide probes or their derivatives which specifically hybridize (e.g. bind) under intracellular conditions, with the cellular mRNA and/or genomic DNA encoding an acetyl glucosaminyl inositol deacetylase polypeptide so as to inhibit expression of that protein or a constituent thereof, e.g. by inhibiting transcription and/or translation. The binding may be by conventional base pair complementarity, or, for example, in the case of binding to DNA duplexes, through specific interactions in the major groove of the double helix. In general, “anti-sense” therapy refers to the range of techniques generally employed in the art, and includes any therapy that relies on specific binding to oligonucleotide sequences.

An anti-sense construct of the present invention can be delivered, for example, as an expression plasmid which, when transcribed in the transformed cell, produces RNA which is complementary to at least a unique portion of the cellular mRNA which encodes an acetyl glucosaminyl inositol deacetylase polypeptide. Alternatively, the anti-sense construct is an oligonucleotide probe which is generated ex vivo and which, when introduced into the cell causes inhibition of expression by hybridizing with the mRNA and/or genomic sequences encoding one of the subject acetyl glucosaminyl inositol deacetylase proteins. Such oligonucleotide probes are preferably modified oligonucleotides that are resistant to endogenous nucleases, e.g. exonucleases and/or endonucleases, and is therefore stable in vivo. Exemplary nucleic acid molecules for use as anti-sense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in anti-sense techniques have been reviewed, for example, by van der Krol et al. (1988) Biotechniques 6:958-976; and Stein et al. (1988) Cancer Res 48:2659-2668.

In addition, the oligomers of the invention may be used as reagents to detect the presence or absence of the target DNA or RNA sequences to which they specifically bind. Such diagnostic tests are described in further detail below.

This invention also provides expression vectors comprising a nucleotide sequence encoding a member of the invention family of acetyl glucosaminyl inositol deacetylase polypeptides and operably linked to at least one regulatory sequence. Operably linked is intended to mean that the nucleotide sequence is linked to a regulatory sequence in a manner that allows expression of the nucleotide sequence. Regulatory sequences are art-recognized and are selected to direct expression of the peptide having an activity of an acetyl glucosaminyl inositol deacetylase polypeptide. Accordingly, the term regulatory sequence includes promoters, enhancers and other expression control elements. Exemplary regulatory sequences are described in Goeddel; Gene Expression Technology: Methods in Enzymology 185, Academic Press, San Diego, Calif. (1990). For instance, any of a wide variety of expression control sequences-sequences that control the expression of a DNA sequence when operatively linked to it may be used in these vectors to express DNA sequences encoding the acetyl glucosaminyl inositol deacetylase polypeptides of this invention. Such useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

As will be apparent, the subject gene constructs can be used to cause expression of the subject acetyl glucosaminyl inositol deacetylase polypeptides in cells propagated in culture, e.g. to produce proteins or peptides, including fusion proteins or peptides, for purification.

This invention also pertains to a host cell transfected with a recombinant acetyl glucosaminyl inositol deacetylase gene in order to express a polypeptide having an activity of an acetyl glucosaminyl inositol deacetylase polypeptide. The host cell may be any prokaryotic or eukaryotic cell. For example, an acetyl glucosaminyl inositol deacetylase polypeptide of the present invention may be expressed in bacterial cells such as E. coli, insect cells (baculovirus), yeast, or mammalian cells. Other suitable host cells are known to those skilled in the art.

Another aspect of the present invention concerns recombinant acetyl glucosaminyl inositol deacetylase polypeptides that have the deacetylase activity of an acetyl glucosaminyl inositol deacetylase polypeptide, or which are naturally occurring mutants thereof. The term “recombinant protein” refers to a protein of the present invention which is produced by recombinant DNA techniques, wherein generally DNA encoding the acetyl glucosaminyl inositol deacetylase polypeptide is inserted into a suitable expression vector which is in turn used to transform a host cell to produce the heterologous protein. Moreover, the phrase “derived from”, with respect to a recombinant gene encoding the recombinant acetyl glucosaminyl inositol deacetylase polypeptide, is meant to include within the meaning of “recombinant protein” those proteins having an amino acid sequence of a native acetyl glucosaminyl inositol deacetylase polypeptide, or an amino acid sequence similar thereto which is generated by mutations including substitutions and deletions of a naturally occurring acetyl glucosaminyl inositol deacetylase polypeptide of a organism.

The present invention further pertains to methods of producing the subject acetyl glucosaminyl inositol deacetylase polypeptides. For example, a host cell transfected with expression vector encoding one of the subject acetyl glucosaminyl inositol deacetylase polypeptide can be cultured under appropriate conditions to allow expression of the peptide to occur. The peptide may be secreted and isolated from a mixture of cells and medium containing the peptide. Alternatively, the peptide may be retained cytoplasmically and the cells harvested, lysed and the protein isolated. A cell culture includes host cells, media and other byproducts. Suitable media for cell culture are well known in the art. The peptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for particular epitopes of the subject acetyl glucosaminyl inositol deacetylase polypeptides.

Thus, a nucleotide sequence derived from the cloning of an acetyl glucosaminyl inositol deacetylase polypeptide of the present invention, encoding all or a selected portion of the protein, can be used to produce a recombinant form of the protein via microbial cellular processes.

The recombinant acetyl glucosaminyl inositol deacetylase polypeptide can be produced by ligating the cloned gene, or a portion thereof, into a vector suitable for expression in bacterial cells. Expression vehicles for production of a recombinant acetyl glucosaminyl inositol deacetylase polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

A number of vectors exist for the expression of recombinant proteins in yeast. For instance, YEP24, YIP5, YEP51, YEP52, pYES2, and YRP17 are cloning and expression vehicles useful in the introduction of genetic constructs into S. cerevisiae (see, for example, Broach et al. (1983) in Experimental Manipulation of Gene Expression, ed. M. Inouye Academic Press, p. 83, incorporated by reference herein). These vectors can replicate in E. coli due the presence of the pBR322 ori, and in S. cerevisiae due to the replication determinant of the yeast 2 micron plasmid. In addition, drug resistance markers such as ampicillin can be used.

The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press: 1989) Chapters 16 and 17. In some instances, it may be desirable to express the recombinant acetyl glucosaminyl inositol deacetylase polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

This invention further contemplates a method of generating sets of combinatorial mutants of the present acetyl glucosaminyl inositol deacetylase polypeptides, as well as truncation mutants, and is especially useful for identifying potential variant sequences (e.g. homologs) that are functional in cleaving acetyl glucosaminyl inositol or other acyl glucosaminyl inositol amide molecules. In a representative embodiment of this method, the amino acid sequences for a population of acetyl glucosaminyl inositol deacetylase polypeptide homologs are aligned, preferably to promote the highest homology possible. Such a population of variants can include, for example, homologs from one or more species, or homologs from the same species but which differ due to mutation. Amino acids that appear at each position of the aligned sequences are selected to create a degenerate set of combinatorial sequences. The presence or absence of amino acids from an aligned sequence of a particular variant is relative to a chosen consensus length of a reference sequence, which can be real or artificial. In order to maintain the highest homology in alignment of sequences, deletions in the sequence of a variant relative to the reference sequence can be represented by an amino acid space (*), while insertional mutations in the variant relative to the reference sequence can be disregarded and left out of the sequence of the variant when aligned.

Further expansion of the combinatorial library can be made by, for example, by including amino acids that would represent conservative mutations at one or more of the degenerate positions. Inclusion of such conservative mutations can give rise to a library of potential acetyl glucosaminyl inositol deacetylase sequences. Alternatively, amino acid replacement at degenerate positions can be based on steric criteria, e.g. isosteric replacement, without regard for polarity or charge of amino acid sidechains. Similarly, completely random mutagenesis of one or more of the variant positions can be carried out.

In a preferred embodiment, the combinatorial acetyl glucosaminyl inositol deacetylase library is produced by way of a degenerate library of genes encoding a library of polypeptides which each include at least a portion of potential acetyl glucosaminyl inositol deacetylase polypeptide sequences. For instance, a mixture of synthetic oligonucleotides can be enzymatically ligated into gene sequences such that the degenerate set of potential acetyl glucosaminyl inositol deacetylase nucleotide sequences are expressible as individual polypeptides, or alternatively, as a set of larger fusion proteins (e.g. for phage display) containing the set of acetyl glucosaminyl inositol deacetylase polypeptide sequences therein.

There are many ways by which the library of potential acetyl glucosaminyl inositol deacetylase homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential acetyl glucosaminyl inositol deacetylase sequences. The synthesis of degenerate oligonucleotides is well known in the art (see for example, Narang, SA (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477.

A wide range of techniques are known in the art for screening gene products of combinatorial libraries made by point mutations, and, for that matter, for screening cDNA libraries for gene products having a certain property. Such techniques will be generally adaptable for rapid screening of the gene libraries generated by the combinatorial mutagenesis of acetyl glucosaminyl inositol deacetylase homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene product was detected. Each of the illustrative assays described below are amenable to high throughput analysis as necessary to screen large numbers of degenerate sequences created by combinatorial mutagenesis techniques.

The invention also provides for reduction of the subject acetyl glucosaminyl inositol deacetylase polypeptides to generate mimetics, e.g. peptide or non-peptide agents, which are able to mimic binding of the authentic acetyl glucosaminyl inositol deacetylase polypeptide to a substrate molecule. Such mutagenic techniques as described above, as well as the thioredoxin system, are also particularly useful for mapping the determinants of an acetyl glucosaminyl inositol deacetylase polypeptide which participate in protein-protein interactions involved in, for example, binding of the subject acetyl glucosaminyl inositol deacetylase polypeptide to a substrate. To illustrate, the critical residues of a subject acetyl glucosaminyl inositol deacetylase polypeptide which are involved in molecular recognition of substrate can be determined and used to generate acetyl glucosaminyl inositol deacetylase-derived peptidomimetics which cleave acetyl glucosaminyl inositol or other acyl glucosaminyl inositol amide substrates and, like the authentic acetyl glucosaminyl inositol deacetylase polypeptide, cleave the substrate molecule, for example by amide hydrolase activity. By employing, for example, scanning mutagenesis to map the amino acid residues of a particular acetyl glucosaminyl inositol deacetylase polypeptide involved in binding to a substrate, peptidomimetic compounds (e.g. diazepine or isoquinoline derivatives) can be generated which mimic those residues cleaving substrate. For instance, non-hydrolyzable peptide analogs of such residues can be generated using benzodiazepine (e.g., see Freidinger et al; in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), beta-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

The invention will be further described with reference to the following examples; however, it is to be understood that the invention is not limited to such examples.

### EXAMPLE 1

Preparation of Reagents and Assay Methods Used. Mycothiol (MSH) was isolated from M. smegmatis and derivatized with monobromobimane to form the MSH-bimane derivative (MSmB).

Mycothiol can be purified from Streptomyces clavuligerus, M. smegmatis, or from cells of any other mycothiol containing bacterium. Early stationary phase cells (25 gm wet weight) were extracted with 250 ml of 50% acetonitirile containing 25 mM methanesulfonic acid and 1 mM DTT at 60° C. for 15 min. The cell debris was removed by centrifugation for 30 min (4° C.) at 20,000×g. The excess acetonitrile was removed using a rotary evaporator. The aqueous extract was neutralized with Tris base to pH 8.0 and clarified by centrifugation for 30 min (4° C.) at 20,000×g The extract was loaded on a 25×70 mm propyl thiol Sepharose 6B resin activated with 2-mercaptopyridine with 1 mmole total thiol binding capacity. The column was washed with 10 column volumes of 20 mM Tris-HCl pH 8.0 to remove unbound extract components. The bound thiols were eluted with 200 ml of 20 mM Tris-HCl pH 8.0 containing 3 mM DTT. The eluate was extracted 5 times with an equal volume of ethyl acetate to remove excess DTT. The ethyl acetate washed aqueous extract was reduced to dryness by lyophilization. The extract was suspended in 5 ml of aqueous 0.1% trifluoroacetic acid (TFA). The mycothiol was purified by preparative HPLC using a 10×250 mm Vydac C-18 (Cat. # 218TP152022) reverse phase column (or equivalent). Mycothiol eluted at 26 min using an isocratic elution in aqueous 0.1% TFA at 5 ml per min. The volatile solvent was removed by lyophilization to yield pure mycothiol. Mycothiol is stable for years when stored as a mildly acidic aqueous solution of 30-100 mM at −70° C.

The bimane derivative of mycothiol is readily made using monobromobimane (Molecular Probes) at 2 mM excess over the thiol concentration. The derivatization is carried out in aqueous 20 mM Tris-HCl pH 8.0 for 15 min in the dark at room temperature. The derivatization mixture was acidified to pH<3 with TFA and extracted 2 times with equal volumes of dichloromethane to remove excess monobromobimane. The derivatization mixture was purified on the preparative reverse phase column described above using a gradient elution. The A-buffer was 0.1% aqueous TFA and the B buffer was methanol. The linear gradient was 15-30% methanol over 30 min with a flow rate of 5 ml per min. The MSmB was detected at 385 nm and eluted as a symmetrical peak at 33 min. The volatile solvent was removed by lyophilization to yield>98% pure MSmB. This derivative was stored as a 10-30 mM solution in water at −70° C.

The purified M. smegmatis mycothiol S-conjugate amidase was used to quantitatively hydrolyze MSMB to stereochemically pure GlcN-Ins, and the latter was purified from the other hydrolysis product, AcCySmB, using a Sep-Pak® C18 (Waters) cartridge. A 10 mM stock solution of GlcNAc-Ins was prepared by addition of a tenfold excess of acetic anhydride (Fisher) to 10 mM GlcN-Ins in 10 mM NaHCO₃ over 20 min while adjusting the pH to 8.5 with NaOH. The reaction was monitored for GlcN-Ins loss as described below to insure that the reaction was complete and that no residual GlcN-Ins was present. This stock solution of GlcNAc-Ins was assayed as described below and used without further purification.

Preparation of Mycothiol S-conjugates. Mycothiol S-conjugates were prepared by reaction of excess electrophile with mycothiol followed by removal of excess electrophile. Stock solutions (100 mM) of iodoacetamide (Sigma) or bromoacetophenone (Sigma) were prepared in acetonitrile. Reaction of these electrophiles at 2 mM in 25 mM HEPES chloride pH 7.5 was initiated by addition of mycothiol to 1 mM from a 32 mM stock solution in H₂O (pH˜3) and was allowed to proceed 15 min at 22° C. Excess reagent was removed by extracting 3 times with an equal volume of H₂O-saturated dichloromethane. Prior to extraction a 1 μl aliquot was withdrawn for reaction with mBBr and analysis of residual mycothiol by HPLC (see above) to verify complete reaction. This showed that>99% of the mycothiol had reacted. The MS-acetophenone was purified to homogeneity by reverse phase preparative HPLC as described above for MSmB, except that MS-acetophenone was detected at 260 nm.

Assay of MSH and MSH precursors. Mycothiol and its precursors cysteine, and Cys-GlcN-Ins were assayed as the bimane derivative by high performance liquid chromatography (HPLC) on a Ultrasphere® ODS IP (Beckman) reversed phase column. The bimane derivatives were detected by fluorescence using a Fluoromonitor III® monitor (Thermo Separation products) with excitation filters 340-380 nm and emission filters 418-700 nm. The A buffer was 0.1% TFA and the B buffer was 7.5% methanol in acetonitrile. The separation was achieved at 22° C. and a flow rate of 1 ml/min with linear gradients as follows: time 0 min 0% B, 10 min 0% B, 30 min 20% B, 33 min 100% B, 36 min 100% B, 38 min 0% B, 50 min reinject. CySmB-GlcN-Ins, CySmB and mycothiol-mB (MSmB) eluted at 28, 31 and 33 min, respectively.

GlcN-Ins was assayed by precolumn derivatization using AccQFluor® (Waters) reagent followed by HPLC analysis. A sample, 7.5 μl of 50% acetonitrile sample containing 5 mM NEM or 5 mM NEM and 1 mM 1,10 phenanthroline was mixed with 39.4 μl of buffer stock. The buffer stock was composed of 30% acetonitrile in aqueous 160 mM HEPES pH 7.5. This was mixed with 15.6 μl of AccQ-Fluor® reagent (Waters) and incubated for 1 min at 22° C. followed by 10 min at 60° C. The sample was diluted with 188 μl of water and the sample was analyzed by HPLC. The sample was centrifuged for 3 min 14,000×g in a microcentrifuge. The HPLC separation conditions were the same as described above for bimane derivatives, except the B buffer was 0.1% TFA in methanol. The product was detected by fluorescence using 254 nm excitation and 370-700 nm emission filters. AccQ-GlcN and AccQ-GlcN-Ins eluted at 12 and 24 min respectively.

Mycothiol S-conjugate amidase assay. The enzymatic activity was routinely assayed by quantitation of the bimane derivative of N-acetylcysteine (AcCySmB) produced from the bimane derivative of mycothiol (MSmB), prepared from purified mycothiol (see above). To conduct an amidase assay a sample (2-10 μL) of extract was mixed with 40 μL of 30 μM MSmB in 3 mM 2-mercaptoethanol, 25 mM HEPES chloride pH 7.5 and reacted 10-30 min at 30° C. before quenching the reaction with 50 μL of 40 mM methanesulfonic acid on ice. The mixture was centrifuged for 3 min at 14,000×g in a microcentrifuge at room temperature and the supernatant analyzed by HPLC without dilution. Separation of MSmB and AcCySmB was achieved by HPLC on a 0.46×250 mM Ultrasphere®ODS column (Beckman) using a linear gradients: time 0 min 10% B, 15 min 18% B, 30 min 27% B, 32 min 100% B, 34 min 10% B, 45 min reinject. Buffer A (aqueous 0.25% acetic acid titrated to pH 3.6 with NaOH) and buffer B (methanol) were pumped at 1.2 ml/min at ambient temperature (22° C.). The bimane derivative of mycothiol (MSmB) eluted at 23.5 min and AcCySmB eluted at 27 min.

### EXAMPLE 2

Cloning and Expression of the Rv1170 gene from M. tuberculosis and demonstration of its GlcNAc-Ins deacetylase activity. Since the invention deacetylases and the mycothiol S-conjugate amidase both cleave an amide bond involving glucosamine, further studies were conducted to determine whether these proteins might be structurally related. A search was conducted of the Sanger Centre database for homologs of the 288 amino acid M. tuberculosis deacetylase, Rv1082. The closest homolog found was Rv1170, with a length of 304 residues and 36% identity to Rv1082 with homology throughout the sequence.

To isolate the Rv1170 gene from M. tuberculosis H37Rv genomic DNA was prepared using known methods. The open reading frame Rv1170 was amplified from this DNA with the following primers:

| | |
|---|---|
| 5′-TAGCCATGGTGTCTGAGACGCCGCG-3′ | (SEQ ID NO:8) |
| and | |
| 5′-GGATCCCGGGGTGAAGCCCAGAC-3′ | (SEQ ID NO:9) |

These primers contain NcoI and BamHI restriction sites, respectively. PCR was performed with Taq polymerase (Gibco BRL), using 1.5 mM MgCl₂ and 5% dimethyl sulfoxide. The 30 cycles of PCR included denaturation at 94° C. for 40 s, annealing at 55° C. for 1 min, and amplification at 72° C. The PCR products were separated on a 1% agarose gel. The appropriate PCR product was ligated into vector pCR2.1 of the TA cloning kit (Invitrogen) and transformed into Escherichia coli DH5α by standard chemical transformation procedures. Clones containing the vector were selected on plates of Luria-Bertani (LB) agar plus ampicillin (100 μg/ml), and plasmid DNA was digested with the restriction endonucleases NcoI and BamHI (Fermentas).

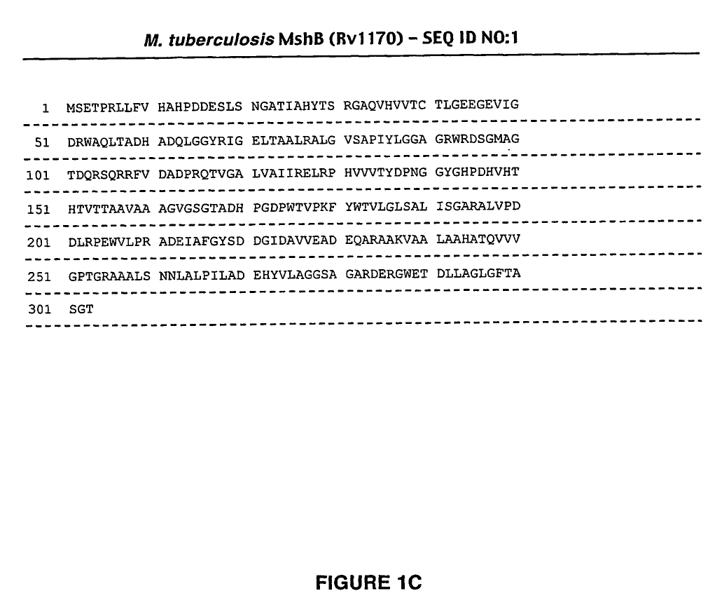

Restriction enzyme-digested plasmids were isolated with a QIAquick® gel extraction kit (Qiagen Ltd.). A corresponding digestion was applied to the plasmid pET-22b, and the two products were ligated together with T4 DNA ligase to obtain the plasmid pYA1170. In order to express Rv1170 in E. coli without the pelB leader sequence, the gene from pYA1170 was excised using NcoI and Bpu1102I (Fermentas) and ligated to an aliquot of pET16b cut with NcoI and Bpu1102I to generate the plasmid pYA1170b (FIG. 2). This plasmid was transformed by the heat shock method to competent E. coli BL21(DE3) prepared according to the CaCl₂ method and plated on LB agar containing 100 μg ampicillin/ml. Single colonies were inoculated into 5 ml of LB broth also containing ampicillin (100 μg/ml). After overnight incubation at 37° C. with shaking, the individual cultures were diluted 1:100 in the same medium and incubation was continued at 37° C. with shaking. Isopropyl-β-D-thiogalactopyranoside was added to a final concentration of 0.4 mM when the A₆₀₀ reached 0.6, and incubation was continued overnight at 25° C. before harvesting by centrifugation at 5000×g for 15 min at room temperature. The pellets were lysed by sonication. Proteins were separated by centrifugation (15,000×g, 4° C., 30 min) into soluble and insoluble fractions. Total proteins were separated by sodium dodecyl sulfate 7.5% polyacrylamide gel electrophoresis (SDS-PAGE) and stained with Coomassie blue or transferred to polyvinylidene difluoride membranes (Bio-Rad). The N-terminal amino acid sequence was verified using Edman degradation after separation of samples by SDS-PAGE and electroblotting to a polyvinylidene difluoride membrane.

E. coli BL21 (DE3) was transformed with the blank cloning vector (pET16b) or with vector containing Rv1170 (pYA1170b). SDS-PAGE was used to separate desalted 20% to 50% saturated ammonium sulfate extracts of these isopropyl-β-D-thiogalactopyranoside induced E. coli. Bio-Rad Broad Range molecular mass standards and purified M. smegmatis mycothiol S-conjugate deacetylase were also assayed for controls.

The results of the SDS-PAGE showed that the 20% to 50% saturated ammonium sulfate fraction from E. coli carrying the Rv 1170 gene contained elevated levels of a protein of the expected size (36 kDa) as compared to those from E. coli prepared with a blank cloning vector. Another, smaller protein (˜26 kDa) was also present at elevated levels and is believed to be a degradation product of the deacetylase. The identity of the 36-kDa protein was confirmed by determining the N-terminal amino acid sequence and finding it identical to that predicted for the protein encoded by open reading frame Rv1170.

The amidase and deacetylase activity of the 20 to 50% saturated ammonium sulfate extracts of E. coli transformed with pYA1170b or with the blank cloning vector pET16b after desalting and concentrating was assayed as described above. The results of these assays are shown in Table 1 below.

TABLE 1
Substrate specificity of crude deacetylase
from E. coli transformed with pYA1170b

Activity (nmol min⁻¹ mg of
protein⁻¹)(n = 3)

| Substrate | Rv1170 | Blank vector |
|---|---|---|
| MsmB^{a} | 1.2 ± 0.1 | <0.002 |
| MSmB^{b} | <10⁻⁴ | ND^{f} |
| GlcNAc-Ins^{c} | 27 ± 2 | <0.002 |
| GlcNAc^{c} | 0.087 ± 0.005 | <0.005 |
| MSH^{d} | 0.002 ± 0.001 | ND |
| MSH^{e} | <10⁻⁴ | ND |

^{a}Analysis for AcCySmB produced by amidase cleavage.
^{b}Analysis for production of the deacetylase product CySmB-GlcN-Ins.
^{c}Analysis for corresponding amine resulting from deacetylase reaction.
^{d}Analysis for AcCys produced by amidase cleavage.
^{e}Analysis for production of the deacetylase product Cys-GlcN-Ins.
^{f}Not determined.

As shown by the results summarized in Table 1, extract from cells expressing Rv1170 exhibited substantial deacetylase activity with GlcNAc-Ins as the substrate, over 300-fold greater than the deacetylase activity determined with GlcNAc and 23-fold greater than the amidase activity measured with MSMB (FIG. 2). Thus, Rv1170 was putatively identified as the GlcNAc-Ins deacetylase gene in the MSH biosynthesis pathway of M. tuberculosis.

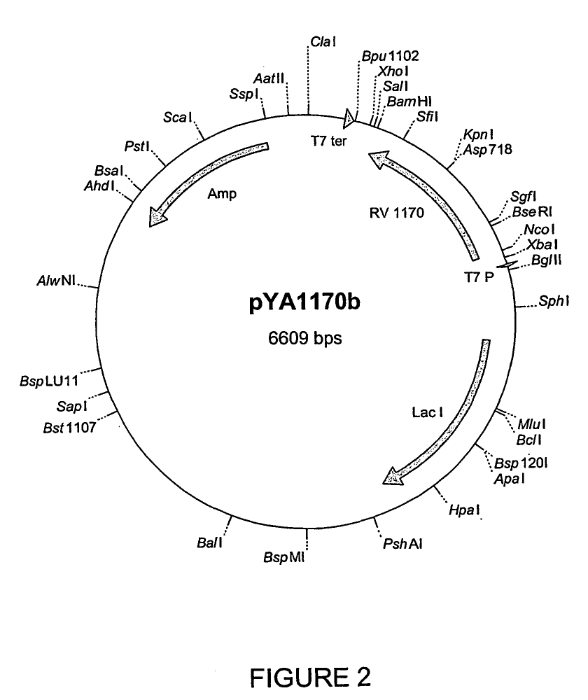

M. smegmatis mycothiol S-conjugate amidase lacks deacetylase activity. Mycothiol S-conjugate amidase from M. smegmatis, a homolog of the protein encoded by M. tuberculosis Rv1082, cleaves the amide bond linking Cys to GlcN in MSH derivatives having an alkylated sulfur residue (G. L. Newton et al., Biochemistry 39:10739-10746, 2000). Tests were conducted to determine whether the mycothiol S-conjugate amidase also cleaves the corresponding bond in GlcNAc-Ins, functioning as a deacetylase (FIG. 3).

GlcNAc-Ins was prepared from GlcN-Ins and the ability of the amidase purified from M. smegmatis to hydrolyze GlcNAc-Ins (i.e., to function as a deacetylase) was compared with its ability to function as an amidase by cleaving the amide bond in the monobromobimane conjugate of MSH (MSmB). The assays utilized are described above. For a test with 100 μM substrate, the activity measured with deacetylase substrate MSmB was 4.5±1.0 μmol min⁻¹ mg of protein⁻¹, whereas that measured with the invention deacetylase substrate GlcNAc-Ins was <1 nmol min⁻¹ mg of protein⁻¹ (n=4). The comparative assays showed that the mycothiol S-conjugate deacetylase does not exhibit measurable deacetylase activity and cannot serve this function in the MSH biosynthesis pathway of M. smegmatis.

### EXAMPLE 3

Extracts of M. smegmatis and mutant 49 have deacetylase activity. If the deacetylase is involved in MSH biosynthesis and if mutant 49 is blocked at an earlier step in the MSH-production pathway, then strains mc²155 and 49 should both produce deacetylase activity. To test this hypothesis, centrifuged extracts of exponentially growing cells were assayed in duplicate for GlcN-Ins production with and without the addition of 100 μM GlcNAc-Ins. The background rate for production of GlcN-Ins measured without added GlcNAc-Ins was high for strain mc²155 (6.1±0.1 pmol min⁻¹ mg of protein⁻¹) presumably reflecting the substantial endogenous level of GlcNAc-Ins present in the undialyzed extract. Addition of 100 μM GlcNAc-Ins increased the rate to 19.7±0.4 pmol min⁻¹ mg of protein⁻¹, giving a net rate increase of 13.6±0.5 pmol min⁻¹ mg of protein⁻¹. For mutant 49, which does not contain GlcNAc-Ins, the background rate was <0.06 pmol min⁻¹ mg of protein⁻¹ and the rate with 100 μM GlcNAc-Ins was 16.4±1.4 pmol min⁻¹ mg of protein⁻¹. Thus, the deacetylase activity of strain 49 is essentially the same as that of the parent strain.

### EXAMPLE 4

Assay for GlcNAc-Ins. To confirm that GlcNAc-Ins is present in mycobacteria, an assay for this component was needed. The availability of cloned Rv1170 allowed the development of such an assay. Cell extracts were prepared in warm 50% acetonitrile containing NEM, as employed previously for an assay of GlcN-Ins (Anderberg et al. (1998) J. Biol. Chem. 273:30391-30397). One portion of the extract was assayed directly for GlcN-Ins content while a second portion was assayed after treatment with a preparation of expressed Rv1170 under conditions sufficient to convert all GlcNAc-Ins to GlcN-Ins. The difference in these two assay results yields the GlcNAc-Ins content for the sample. In most cases, GlcN-Ins levels were at least an order of magnitude lower than those of GlcNAc-Ins, so this method gives reliable results.

Recovery experiments for GlcNAc-Ins were conducted using a M. smegmatis MSH-deficient mutant (“mutant 49”), derived from mc²155 by chemical mutagenesis. Mutant 49 was cultured in Middlebrook 7H9 medium at 37° C. as previously described (Newton, et al. (1999) Biochem. Biophys. Res. Comm. 255:239-244).

In the uptake and metabolism assay, 50% acetonitrile-NEM extracts were analyzed after the addition of 0.7 and 7.0 μM GlcNAc-Ins. Extracts of mutant 49 contained no detectable GlcN-Ins or MSH, so that any GlcN-Ins found in the extracts after treatment with the deacetylase preparation was derived solely from the deacetylation of GlcNAc-Ins.

The relationship between A₆₀₀ and RDW was determined for aliquots of mc²155 cells producing ˜30 mg of RDW and found to be 0.42±0.02 mg of RDW per ml of cells with A₆₀₀ at 1.00. No variation of >15% in this value was seen between log- and stationary-phase cells.

The method was tested with extracts prepared from log-phase cells of mutant 49 by spiking the extract with GlcNAc-Ins at levels equivalent to 0.13 and 0.013 μmol per g of RDW. The recoveries from triplicate determinations were 97%±5% and 132%±3%, respectively. At the lowest level of added GlcNAc-Ins, a significant correction had to be applied for background peaks present in unspiked controls that overlapped the GlcN-Ins peak. The greater than 100% recovery found for the samples spiked at lowest level indicates that this correction produces a systematic error and thus defines the lower limit of useful sensitivity for this assay.

### EXAMPLE 5

M. smegmatis mc²155 produces GlcNAc-Ins but MSH-deficient mutant 49 does not. M. smegmatis mc²155 cells were examined in exponential and stationary phases for their content of MSH and its precursors using the methods described above. The results of these studies are summarized in Table 2 below:

TABLE 2
Levels of MSH and precursors in M. smegmatis mc²155

| | | Content (μmol [g of RDW]⁻¹) ( n = 3) | | |
|---|---|---|---|---|
| Growth phase | A₆₀₀ | MSH | GlcNAc-Ins | GlcN-Ins |
| Log | 0.54 | 7.8 ± 0.6 | 14.5 ± 1.2 | 0.2 ± 0.1^{a} |
| Stationary | 3.3 | 6.6 ± 0.6 | 12.6 ± 0.6 | <0.03 |

^{a}Best estimate after correction for overlapping peak in HPLC analysis.

These results show that M. smegmatis accumulates GlcNAc-Ins to a substantial level, almost twice the level of MSH content. The GlcN-Ins content was very much lower in log phase and declined further in stationary phase, as had been observed previously (Anderberg et al; (1998) Supra.

### EXAMPLE 6

MSH-deficient mutant 49 can import GlcNAc-Ins and utilize it to sythesize MSH. The foregoing results imply that mutant 49 should be able to synthesize MSH if supplied with GlcNAc-Ins. The concentration dependence of the uptake of GlcNAc-Ins was examined with cultures of M. smegmatis mutant 49. For this study mutant 49 was cultured at 37° C. for 23 hr in 5 ml of 7H9 Middlebrook medium as described below, supplemented with 0, 4, 8, 21 μM GlcNAc-Ins. The cells were collected by centrifugation and extracted for amine and thiol analysis as described above. The absence of GlcNAc-Ins in mutant 49 shows that it is defective in an initial step of MSH biosynthesis. When mutant 49 was grown in media supplemented with GlcNAc-Ins, the cells did show the presence of MSH and mycothiol precursors in increasing concentrations with the media content of GlcNAc-Ins. These results established that GlcNAc-Ins is indeed imported intact, deacetylated and utilized for the production of MSH in a concentration dependent manner (Table 3).

TABLE 3
Levels of MSH and precursors in MSH-deficient strain 49 (M. smegmatis)

| | Content (μmol [g of RDW]⁻¹⁾ | | |
|---|---|---|---|
| [GlcNAc-Ins]^{a} (μM) | MSH | GlcNAc-Ins | GlcN-Ins |
| 0 | <0.04 | <0.01 | <0.02 |
| 4 | 0.47 | 0.39 | <0.02 |
| 8 | 0.91 | 0.80 | 0.026 |
| 21 | 2.1 | 1.4 | 0.047 |

^{a}In Middlebrook 7H9 medium.; [GlcNAc-Ins] = 0).

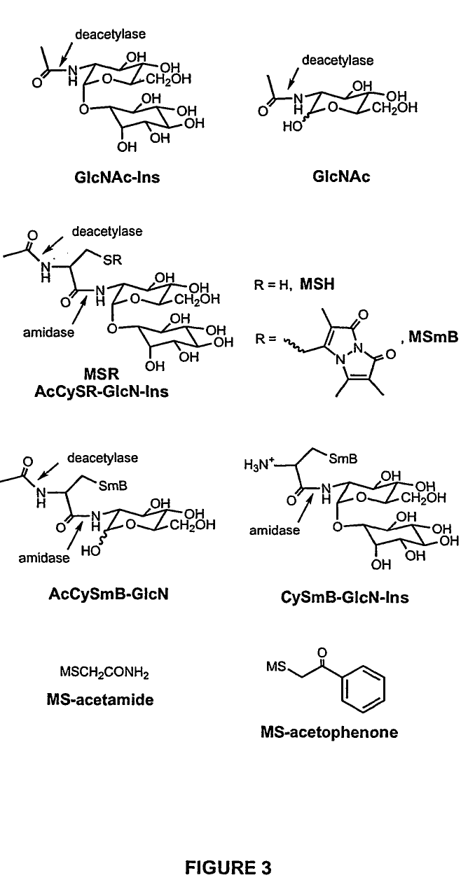

In another experiment mutant 49 was cultured in 7H9 Middlebrook medium with 0.4% glucose and 0.5% TWEEN 80 detergent to log phase (A600=0.6) and 10-ml aliquots transferred to sterile 25 ml erlenmeyer flasks. Duplicate flasks contained cells only (control), 20 μM inositol, 20 μM glucosamine, 20 μM N-acetylglucosamine, or 17 μM GlcNAc-Ins and were cultured at 37° C., shaking at 225 rpm. Samples (2.3×10⁸ cells) were taken at 2, 6, 19 and 46 hr of culture. the cells were pelleted by centrifugation for 2 min at 14,000×g and were extracted in 50% acetonitrile containing 20 mM HEPES (pH 8.0) and either 2 mM monobromobimane for thiol analysis or 5 mM NEM for amine analysis and thiol control samples. Cells from duplicate cultures were analyzed for GlcN-Ins, GlcNAc-Ins, Cys-GlcN-Ins, cysteine, and MSH. No significant MSH was found in any sample except for the cultures supplemented with GlcNAc-Ins (FIG. 4).

Strain 49 cultured in 17 μM GlcNAc-Ins showed a cysteine level that remained unchanged at 0.36±0.08 μmol per gm of residual dry weight over the experiment. Cells contained 2.9 μmole per gm of residual dry weight of GlcNAc-Ins at the first measurement (2 hr), which corresponds to a level of GlcN-Ins approaching millimolar. This occurred prior to the appearance of significant GlcN-Ins in the cell, showing that strain 49 is able to import and concentrate GlcNAc-Ins intact prior to its deacetylation. The level of GlcNAc-Ins appeared to fall at 6 hr before rising to an even higher level in stationary phase.

The MSH content was measurable as early as 2 hr, but was about 10-fold less than the GlcNAc-Ins content at that time. The MSH content increased thereafter, and the level at 48 hr was about 60% of that measured for the parent strain (Table 2). The level of GlcN-Ins ranged from 0.025 (2 hr) to 0.1 (48 hr) μmole per gm of residual dry weight, values in the range of those measured for mc²155 (Table 2). These results show that exogenous GlcNAc-Ins is efficiently imported by strain 49 and substantially restores its defective biosynthesis of MSH. The intracellular level of GlcN-Ins found in mutant 49 at 48 hours indicates that GlcNAc-Ins has been concentrated more than 100 fold from the supplemented medium. This implies that toxic analogs of GlcNAc-Ins, such as those with oxidizing or nitrosating moieties, will be concentrated inside pathogenic mycobacteria or other actinomycetes and function as antimicrobial agents.

### EXAMPLE 7

Cloning, expression and purification of M. tuberculosis His-tagged MshB. To facilitate the purification of the M. tuberculosis deacetylase, Rv1170 was cloned to contain a C-terminal HisTag permitting its easy isolation by affinity chromatography on a Ni²⁺ chelate resin. Genomic DNA of M. tuberculosis H37Rv was prepared as described previously (Av-Gay et al., Infect. Immun. 67:5676-5682, 1999). The open reading frame Rv1170 was amplified from this DNA with the primers

| | |
|---|---|
| 5′-TTCATATGGTGTCTGAGACGCCG-3′; | (SEQ ID NO:10 |
| and | |
| 5′-ATAAGCTTGTAGCCGGACGCGGTG-3′ | (SEQ ID NO:11) |

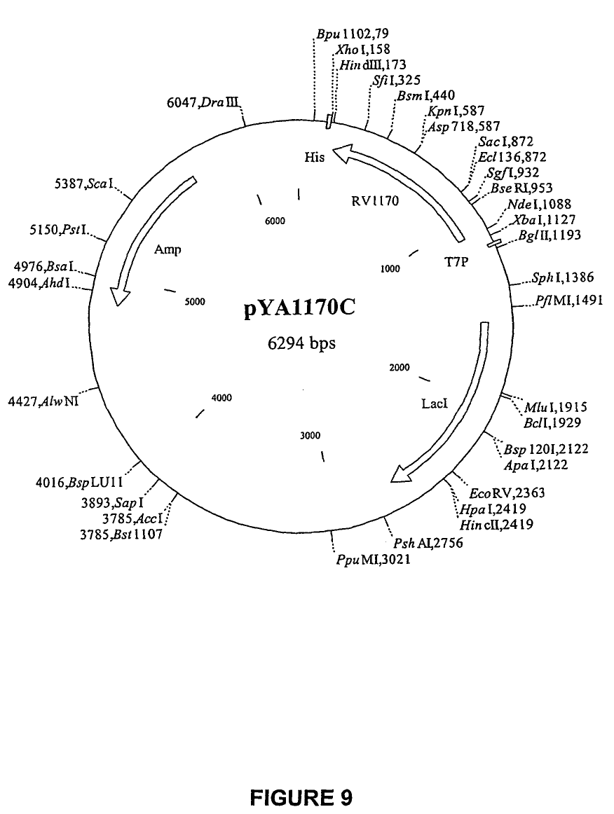

containing NdeI and HindIII restriction sites, respectively. PCR was performed with Taq polymerase obtained from Gibco BRL, using 1.5 mM MgCl₂ and 5% dimethyl sulfoxide. The 30 cycles of PCR included denaturation at 94° C. for 40 seconds, annealing at 55° C. for 1 min and amplification at 72° C. The PCR products were separated on a 1% agarose gel. The appropriate PCR product was ligated into the vector pCR2.1 of the TA cloning kit (Invitrogen) and transformed into E. coli DH5a by standard chemical transformation procedure. Clones containing the vector were selected on plates of Luria-Bertani (LB) agar plus ampicillin (100 μg/ml) and plasmid DNA was digested with restriction endonucleases NdeI and HindIII (Fermentas). Restriction enzyme-digested plasmids were isolated with a QIAquick gel extraction kit (Qiagen Ltd.). A corresponding digestion was applied to the plasmid pET-22b and the two products were ligated together with T4 DNA ligase to obtain the plasmid pYA1170c (shown in FIG. 9). This provides a recombinant protein without a pelB leader sequence, but with a c-terminal His tag. This plasmid was transformed by the heat shock method to competent E. coli BL21(DE3) prepared according to the CaCl₂ method and plated on LB agar containing 100 μg/ml ampicillin. Single colonies were inoculated into 5 ml of LB broth also containing ampicillin (100 μg/ml). After over-night incubation at 37° C. with shaking, 5 1-liter cultures were inoculated to 1% with the starter culture and incubated at 37° C. with shaking. Isopropyl-β-D-thiogalactopyranoside was added to a final concentration of 0.5 mM when the A600 reached 0.6 and incubation was continued overnight at 22° C. before harvesting by centrifugation at 5000×g for 20 minutes at 4° C. The pellets were lysed by sonication in 3 mM 2-mercaptoethanol with 50 mM HEPES pH 7.4 (lysis buffer) containing 35 μM each protease inhibitors (N-α-p-tosyl-L-lysinechloromethyl ketone and N-α-p-tosyl-L-phenylalaninechloromethyl ketone). Proteins were separated by centrifugation (1-5,000×g, 4° C., 30 min) into-soluble and insoluble-fractions. The soluble proteins were applied to a 2.5×11 cm Fast Flow® chelating resin (Pharmacia) in the Ni²⁺ form in lysis buffer containing 10 mM imidazole. The column was washed with 10 volumes of the loading buffer and then step eluted with lysis buffer containing 150 mM imidazole followed by lysis buffer containing 500 mM imidazole. Fractions were collected and the purity of the eluted protein was assayed by SDS PAGE gel electrophoresis. The early 150 mM imidazole fractions were contaminated with E. coli proteins, but the later fractions of the 150 mM and the early 500 mM imidazole fractions were judged>95% pure and pooled. The yield of pure recombinant MshB (Rv1170) was estimated to be ˜125 mg of protein. The protein was concentrated and the buffer exchanged repeatedly with 50 mM HEPES containing 50 mM NaCl (assay buffer) in a Centricon® 10 spin ultra filter, adjusted to 10% glycerol and frozen at −70° C.

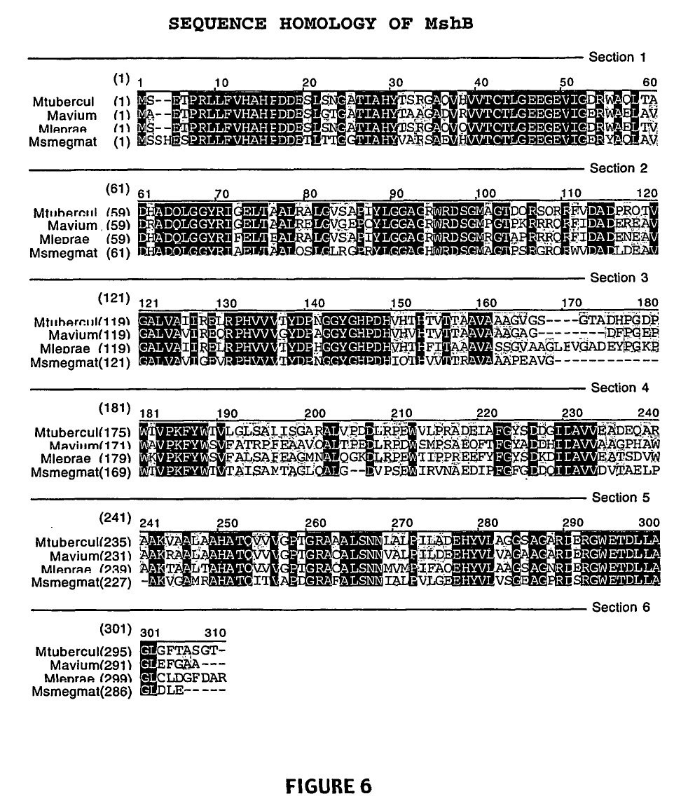

The enzyme as purified was assayed for deacetylase activity with 0.1 mM GlcNAc-Ins as substrate and was found to be sensitive to chelating agents such as 1,10-phenanthroline. The deacetylase reaction was substantially inhibited by the presence of 0.1 mM 1,10-phenanthroline in the reaction mixture (FIG. 7). 1 mM 1,10-phenanthroline completely inhibited the deacetylase reaction, indicating a dependence of the reaction on divalent metal ions.

The apoenzyme was generated by treatment of the concentrated enzyme with 5 mM 1,10-phenanthroline in assay buffer for 30 min on ice. The chelator was removed by repeated dilution with assay buffer and concentration until the 1,10-phenanthroline concentration was estimated to be below 1 μM. All solutions were treated with Chelex-100® chelator to remove extraneous divalent cations. The apoenzyme was stored at 10 mg/ml in 50 mM NaCl+50 mM HEPES pH 7.5 with 10% glycerol at −70° C. The zinc form (Zn²⁺) of the deacetylase was prepared by treating the apoenzyme with 0.1 mM ZnCl₂ in the assay buffer for 30 min on ice. The excess Zn²⁺ was removed by repeated dilution and concentration with assay buffer until the Zn²⁺ was estimated to be below 1 μM. The Zn²⁺ form of the enzyme was stored as described above for the apoenzyme and is herein referred to as “His-tagged MshB”.

### EXAMPLE 8

Metal ion dependence of the deacetylase. The metal ion dependence of the deacetylase activity of MshB was assayed using GlcNAc-Ins in the presence of 0.1 mM divalent metal ion. Previous studies with ZnCl₂ have shown that excess Zn²⁺ is not inhibitory at 0.1 mM and only slightly inhibitory at 1 mM (see above). The metal salts tested, ZnCl₂, CuSO₄, MgCl₂, MnCl₂, CaCl₂, COCl₂, CdCl₂ and NiCl₂, were of reagent grade or higher purity and solutions of the metals were prepared as 10 mM stock solutions in water treated with Chelex-100 chelator. Enzymatic activity was assayed in Chelex-100® treated 50 mM NaCl with 50 mM HEPES pH 7.4 (assay buffer) containing 0.1 mM of the desired metal salt and 0.1 mM GlcNAc-Ins as substrate. Buffer and 10 μg of purified apoenzyme were preincubated the divalent metal ion in an 80 μl total reaction volume for 10 min at 37° C. and the assay initiated by the addition of the substrate. Duplicate reactions were sampled at 3, 10, and 30 min by removing a 20 μl sample of the reaction mixture and adding it to 20 μl of acetonitrile containing 10 mM NEM and 2 mM 1,10-phenanthroline. The samples were incubated for 10 min at 60° C., placed on ice, and subsequently reacted with AccQ Fluor® (Waters) reagent as described above assayed for AccQ-GlcN-Ins by HPLC as described above.

The MshB apoenzyme was able to catalyze both the deacetylase (GlcNAc-Ins as substrate) and the amidase (MSmB as substrate) reactions at substantially reduced rates, as shown in Table 4 below.

TABLE 4
M. tuberculosis MshB (Rv1170) metal ion dependence^{a}

| | Amidase | | | |
|---|---|---|---|---|
| | Specific | | Deacetylase | |
| | Activity^{b} | Amidase | Specific | Deacetylase |
| Metalloenzyme | nmole/ | Activity | Activity^{c} | Activity |
| Form | min/mg | relative rate | nmole/min/mg | Relative rate |
| Apoenzyme | 3.4 ± 0.5 | 1.0 | 35 ± 6 | 1.0 |
| Untreated | 15 ± 0.2 | 4.4 | 70 ± 19 | 2.0 |
| Zn²⁺ | 17 ± 0.7 | 5.0 | 81 ± 16 | 2.3 |
| Ni²⁺ | 13 ± 0.7 | 3.8 | 62 ± 23 | 1.8 |
| Mn²⁺ | 22 ± 0.7 | 6.5 | 69 ± 5 | 2.0 |
| Ca²⁺ | 3.3 ± 0.1 | 0.97 | 34 ± 5 | 0.97 |
| Mg²⁺ | 3.5 ± 0.2 | 1.0 | 41 ± 4 | 1.2 |
| Cu²⁺ | 3.2 ± 0.1 | 0.94 | 32 ± 10 | 0.9 |
| Cd²⁺ | 21 ± 1.5 | 6.2 | 34 ± 2 | 0.97 |
| Co²⁺ | 29 ± 0.3 | 8.5 | 236 ± 23 | 6.7 |
| Zn²⁺ + Mg²⁺ | ND | — | 75 ± 14 | 2.1 |
| Ni²⁺ + Mg²⁺ | ND | — | 64 ± 15 | 1.8 |

^{a}Recombinant Rv1170 purified to homogeneity over Ni²⁺ Sepharose.
^{b}0.1 mM MSmB as substrate with 0.1 mM metal ion.
^{c}0.1 mM GlcNAc-Ins as substrate with 0.1 mM metal ion.

The low metal ion content of the apoenzyme reduced the amidase activity to a greater extent than the deacetylase activity relative to the untreated purified protein. The activity of the Ni²⁺ Sepharose purified (untreated) form of the MshB appears to be consistent with the Ni²⁺ form of the enzyme. The Zn²⁺, Mn²⁺ and Ni²⁺ forms appear to have about the same activity for both the amidase and deacetylase reactions. The MshB apoenzyme was not activated by the presence of Ca²⁺, Mg²⁺ or Cu²⁺ for either the amidase or deacetylase reactions. The MshB deacetylase and amidase reactions were differently affected by the presence of the Cd²⁺ ion. The amidase reaction was enhanced to the level of Zn²⁺ while the deacetylase reaction remained at the apoenzyme activity level. Similarly anomalous activity with Cd²⁺ was observed for the peptidase and esterase activity of carboxypeptidase, a zinc metalloprotease (R. C. Davis et al Biochemistry 7, 1090-1099 (1968)). The highest level of activation for both the amidase and deacetylase reactions was observed with Co²⁺, as has been reported for numerous zinc metalloproteases (D. S. Auld, Chapter 14, Methods in Enzymology, 248:229-243, 1995). Some zinc metalloproteases have been reported to require the presence of Mg²⁺ for optimal activity (Vallee and Auld (1993) Biochemistry 32:6493-6500) so activity assays were conducted with 0.1 mM each Zn²⁺ and Mg²⁺. No additional activation over that of Zn²⁺ alone for either the amidase or deacetylase reactions was observed. Thus, the metal ion dependence of MshB is similar to that found for members of the metalloprotease superfamily and is therefore likely that MshB is a zinc metalloenzyme in its native state.

### EXAMPLE 9

Kinetics and substrate specificity of His-tagged MshB. The kinetic constants were determined for GlcNAc-Ins, the deacetylase substrate for MshB, and for MSmB, an amidase substrate. The rates were determined with His-tagged MshB in 50 mM NaCl containing 50 mM HEPES pH 7.5 (assay buffer). The enzyme (10 μg) was preincubated with 80 μl of assay buffer for 10 min at 37° C. and the reaction was initiated with the addition of the substrate. The deacetylase substrate was assayed at 9 different concentrations between 10 μM and 2 mM. Duplicate reactions were sampled at 3, 10, and 30 min by removing 20 μl of the sample, and mixing with 20 μl of acetonitrile containing 10 mM NEM and 2 mM 1,10-phenanthroline. The samples were quenched by incubation for 10 min at 60° C. The quenched sample was reacted with AccQ Fluor® reagent (Waters) and analyzed for GlcN-Ins by HPLC as described above. The amidase reaction was assayed similarly using 10 concentrations of MSmB between 10 μM and 10 mM. The amidase reaction was sampled at 3, 10, and 30 min by removing 20 μl of sample and mixing it with 60 μl of 40 mM methanesulfonic acid. The mercapturate product, AcCySmB was analyzed by HPLC as described above. Initial rates were obtained by extrapolation of the rates calculated at 3, 10, and 30 min to zero time.

The kinetic constants were obtained from an Eadie-Hofstee plot. The amidase and deacetylase substrates have Km values near of 0.5 mM as shown below in Table 5 below.

TABLE 5
Kinetic characterization of M. tuberculosis MshB (Rv1170)

| | | Kₘ | Vₘₐₓ | k_{cat} |
|---|---|---|---|---|
| Substrate | Activity | μM | nmol min⁻¹mg⁻¹ | s⁻¹ |
| GlcNAc-Ins | deacetylase | 400 | 470 | 0.25 |
| MSmB | amidase | 560 | 220 | 0.12 |

The intracellular concentration of GlcN-Ins is roughly twice the mycothiol level, or about 2-6 mM (Newton et al, J. Bacteriol., 182: 6958-6963, 2000) and is sufficient to saturate the enzyme at physiological concentrations.

The substrate specificity of MshB was examined with various amidase and deacetylase substrates at a concentration of 0.1 mM. The product analyzed will vary with the substrate used. The deacetylase substrates tested were GlcNAc, GlcNAc-Ins, MSH, and MSmB, and the products assayed were GlcN, GlcN-Ins, Cys-GlcN-Ins, and CySmB-GlcN-Ins, respectively. The amidase substrates tested were MSH, MSmB, AcCySmB-GlcN, CySmB-GlcN-Ins, MS-acetamide, and MS-acetophenone and the product assayed was GlcN-Ins, except for AcCySmB-GlcN were GlcN was assayed. The amines were analyzed as the AccQ-derivative and thiols as the bimane derivative as described above. The results of these substrate specificity studies are summarized in Table 6 below.

TABLE 6
Substrate specificity of M. tuberculosis MshB (Rv1170)^{a}

| | Amidase | | |
|---|---|---|---|
| | Specific | Deacetylase | |
| | Activity^{b} | Specific Activity^{b} | Relative |
| Substrate^{a} | nmol min⁻¹mg⁻¹ | nmol min⁻¹mg⁻¹ | Rate |
| GlcNAc-Ins^{c} | | 70 ± 3 | 1.0 |
| GlcNAc^{d} | | 1.6 ± 1 | 0.023 |
| MSH^{e} | | <0.025 ± 0.012 | <0.00036 |
| MSH^{c} | 0.3 ± 0.1 | | 0.0043 |
| MSmB^{f} | | <0.006 | <0.00009 |
| MSmB^{c} | 20 ± 2 | | 0.29 |
| AcCySmB-GlcN^{d} | <0.1 | | <0.001 |
| CySmB-GlcN-Ins^{c} | 265 ± 10 | | 3.8 |
| MS-acetamide^{c} | 0.14 ± 0.013 | | 0.002 |
| MS-acetophenone^{c} | 88 ± 8 | | 1.3 |

^{a}His tagged MshB (Rv1170) in the Zn²⁺ form.
^{b}0.1 mM substrate assayed in 50 mM NaCl + 50 mM HEPES pH 7.4 at 37° C.
^{c}Reaction assayed for GlcN-Ins.
^{d}Reaction assayed for GlcN.
^{e}Reaction assayed for CyS-GlcN-Ins.
^{f}Reaction assayed for CySmB-GlcN-Ins.

As shown by the results in Table 6, the deacetylase activity of MshB is dependent on the presence of the inositol moiety as the GlcNAc substrate is 44 fold less active than GlcNAc-Ins. Interestingly MSH and MSmB are not substrates for the deacetylase activity of MshB. However, mycothiol S-conjugates with reasonably large groups on the cysteine sulfur, such as acetophenone and bimane S-conjugates, are good substrates for the amidase activity of the MshB. The smaller mycothiol S-conjugate acetamide is a poor substrate for the MshB amidase activity, as was observed previously for mycothiol S-conjugate amidase from M. smegmatis (Newton et al Biochemistry, 39, 10739-10746, 2000). The importance of the inositol moiety for the amidase activity of MshB is also apparent by the lack of activity with AcCySmB-GlcN. One difference between the amidase activity of M. tuberculosis His-tagged MshB and the amidase from M. smegmatis is the ability to use CySmB-GlcN-Ins as substrate. The M. smegmatis amidase required the acetylated amine on the cysteine moiety (or MSmB) for activity. Another difference between MshB and the M. smegmatis amidase is that the amidase will not deacetylate GlcNAc-Ins (Newton et al J. Bacteriol., 182, 6958-6963, 2000).

### EXAMPLE 10

Inhibition of His-tagged MshB. Naturally occurring inhibitors of the His-tagged MshB deacetylase and amidase activities have been examined. The inhibition assays are similar to those described above in Example 9. For the amidase reaction of His-tagged MshB 10 μg enzyme was preincubated for 10 min at 37° C. in 80 μl of buffer containing 50 mM NaCl, 50 mM HEPES pH 7.5, 0.1 mM ZnCl₂, 0.2 mM DTT and 0.1-1.0 mM inhibitor. The inhibitors examined were mycothiol, glutathione and GlcN-Ins. The purpose of the excess Zn⁺² is to prevent depletion of the protein bound Zn²⁺ by thiols. The DTT is added to limit the oxidation of the thiol inhibitors, which can be significant at the lower concentrations. The uninhibited enzyme activity was determined using the same buffer and preincubation conditions without inhibitor. The assays were initiated with the addition of MSmB to 0.55 mM and sampled at 3, 10, and 30 min by removing a 20 μl sample and quenching the reaction by mixing with 60 μl of 40 mM aqueous methanesulfonic acid. This sample was assayed directly by HPLC for AcCySmB as described above.

The deacetylase reaction was assayed with GlcNAc-Ins using 0.1-1.0 mM glutathione and mycothiol as inhibitors. The reaction was initiated by the addition of GlcNAc-Ins to 0.8 mM and was sampled at 3, 10, and 30 min. The reaction was quenched and analyzed for GlcN-Ins as described in Example 9.

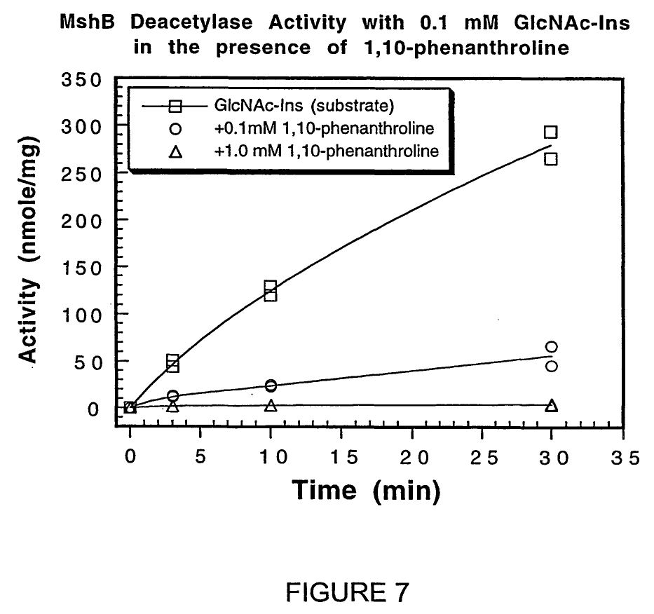

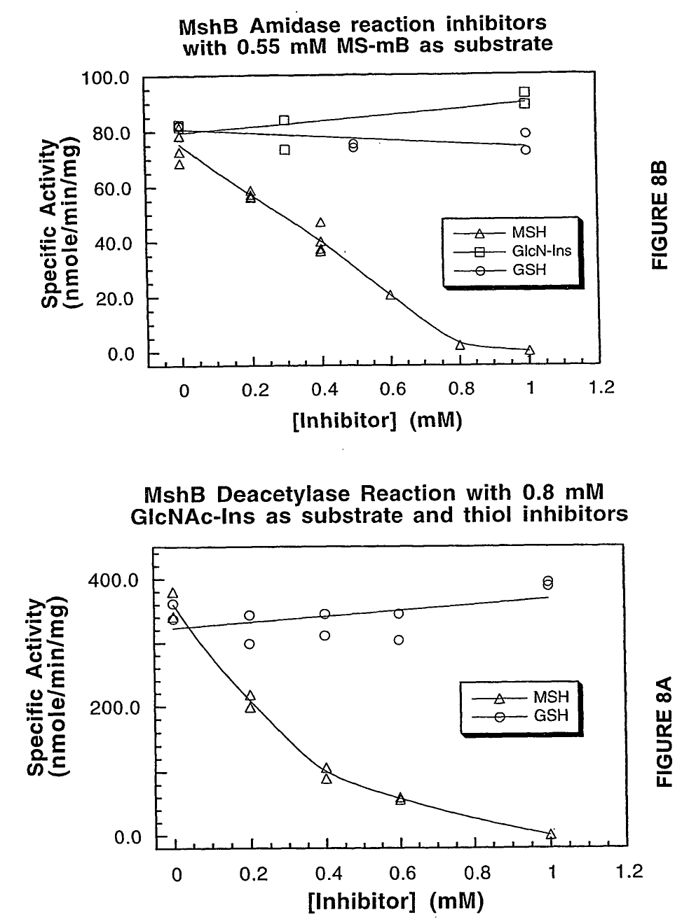

Mycothiol appears to be an effective inhibitor of both the amidase (FIG. 8A) and the deacetylase (FIG. 8B) activities of His-Tagged MshB in the range of 0.1 to 1 mM. This is not simply due to disruption of the enzyme metal binding site by a thiol as shown by the lack of inhibition by glutathione in this concentration range. The lack of inhibition of the amidase reaction by GlcN-Ins (FIG. 8A) also shows that the immediate product of the reaction is not inhibitory. Mycothiol is the terminal product in the biosynthesis scheme shown in FIG. 5 and it appears to completely inhibit the deacetylase (MshB) at mycothiol concentrations above 1 mM. The estimated intracellular concentration of mycothiol in mycobacteria is about 1-3 mM and thus the deacetylase is in a substantially inhibited state until the mycothiol level falls below 1 mM, as may happen during cell division or cell stress. Inhibition of the deacetylase reaction was analyzed at 0.8 mM GlcNAc-Ins, a concentration nearing saturation, but still below the estimated cellular concentration of 2-6 mM for M. smegmatis. Thus, in M. smegmatis the deacetylase is exposed to saturating GlcNAc-Ins concentrations but is inhibited when the cellular mycothiol level is above 1 mM. If mycothiol level falls substantially below 1 mM, the enzyme will catalyze the deacetylation of GlcNAc-Ins from a large cellular pool at near maximal rates to GlcN-Ins and ultimately to mycothiol. It has previously been shown that mycothiol levels are quite constant from logarithmic through stationary phase growth of M. smegmatis (Newton et al, J. Bacteriol., 178: 1990-1995, 1996). It appears that the intracellular mycothiol level is regulated by feedback inhibition of the GlcNAc-Ins deacetylase by mycothiol itself.

The amidase substrate, MSmB, served as a useful model substrate for finding inhibitors of the GlcNAc-Ins deacetylase. The amidase and deacetylase activities are both completely inhibited by mycothiol at concentrations over 1 mM. These results identify mycothiol as an inhibitor for the deacetylase and amidase reactions with similar inhibitory concentrations. Thus, the substrates for either the amidase or deacetylase activity of GlcNAc-Ins deacetylase can be used to screen for inhibitors of the enzyme.

### EXAMPLE 11

Oxidant sensitivity of M. smegmatis MSH-deficient mutants. The importance of mycothiol biosynthesis for the survival of mycobacteria under oxidative stress is illustrated by results obtained with chemical and transposon mutants deficient in mycothiol biosynthesis. Chemical mutants were produced by mutagenesis of M. smegmatis mc²155 (˜2.5×10⁹ colony forming units per ml Middlebrook 7H9 liquid medium) with N-methyl-N-nitro-N-nitrosoguanidine (4 μg per ml) for 30 min at 37° C. Cells were washed three times with 9 ml of fresh medium, resuspended in 10 ml of fresh medium, and incubated 8.5 h at 37° C. with shaking. After dilution in fresh medium and passage through a 22-guage needle, the cells were plated on Middlebrook 7H9 agar containing 0.05% Tween 80 and 0.4% glucose. After 9 days in a humidified incubator, the plates were scored and individual colonies replated in duplicate using a grid layout in 100 mm culture dishes. After colony development, one plate was used to screen for MSH-deficient mutants using a membrane-based immunoassay specific for MSH (Unson, et al. (1998) J. Immunol. Methods 214:29-39).

MSH-deficient candidates were grown in bulk in liquid medium and analyzed for MSH content as described in Example 1. Two MSH-deficient mutants (strains 49 and 164) were identified and strain 49 was complemented using a M. tuberculosis library (pYUB412::H37Rv) of ˜18 kb DNA fragments cloned in the integrative shuttle plasmid pYUB412 to produce the strain 49::H37Rv A18 with partially restored MSH biosynthesis.

Transposon mutants of M. smegmatis mc²155 were prepared using the pCG79 plasmid carrying transposon Tn611 following the methods of Perez, et al. (1998) Meth. Molecular Biol. 101:187-198). Plasmid pCG79 was introduced into M. smegmatis mc²155 by electroporation after which the cells were incubated 2 h at 32° C. in 7H9 medium without antibiotic. They were then plated on Middlebrook 7H10 agar plates with ADC enrichment and 20 μg/mL of kanamycin and incubated 6 days at 32° C. Single colonies were restreaked on 7H10km plates and incubated 4 days at 32° C. A loopful of each streaked transformant was resuspended, serially diluted, and plated in duplicate on 7H10 Km plates. One set of plates was incubated 36 h at 32° C. and then shifted to 42° C. for 5 days to select transposon mutants. The other set of plates was incubated at 32° C. for 7 days and served as positive controls. The transposon mutants were replated on 7H10Km plates at 1000-2000 colonies per plate and incubated 5d at 42° C. The cells were scraped into 10 ml of 7H9 medium, mixed with 1 mL of glycerol, and stored at −70° C. This library contained ˜10,000 mutants. Screening of the library for MSH-deficient mutants was accomplished using as ELISA assay method to detect MSH production in cultures grown in microtiter plates (Unson, et al. (1999) J. Clin. Microbiol 37:2153-2157). The first 1000 mutants screened produced three nearly identical MSH-deficient mutants (TN1, TN2 and TN3) that accumulated GlcN-Ins to high levels but failed to produce detectable levels of Cys-GlcN-Ins or MSH. Since these clones seemed likely to be daughter cells, TN3 was selected for more detailed studies.

Results obtained with the parent and mutant strains of M. smegmatis are summarized in Table 7 below.

TABLE 7
Characterization of M. smegmatis mutant strains

| | MSH Content | | H₂O₂ IC₅₀^{a} |
|---|---|---|---|
| Strain | μmol per g RDW | Enzyme Deficiency | mM |
| mc²155 | 10 | None | 12 |
| 49^{b} | <0.004 | no MshA^{c} | 1 |
| 49::H37Rv A18^{b} | 3.3 | partial MshA^{d} | 1.8 |
| I64 | 0.1 | low MshC^{e} | 3.5 |
| TN3 | <0.004 | no MshC^{e} | 0.5 |

^{a}Concentration producing 50% loss of viability following 2 h incubation in Middlebrook 7H9 liquid medium containing H₂O₂ at 37° C., dilution and plating on 7H10 agar, incubation for 10 d, and counting of colony formation.
^{b}Data from Newton, et al. (1999) Biochem. Biophys. Res. Commun. 255: 239-244.
^{c}Based upon failure to produce GlcNAc-Ins or GlcN-Ins.
^{d}Based upon restoration of ability to produce GlcN-Ins to ˜15% that of the parent strain.
^{e}Based upon absence of MshC activity in cell extracts and accumulation of GlcN-Ins to 2-3 μmol per g RDW (˜25-fold above the level of the parent strain).

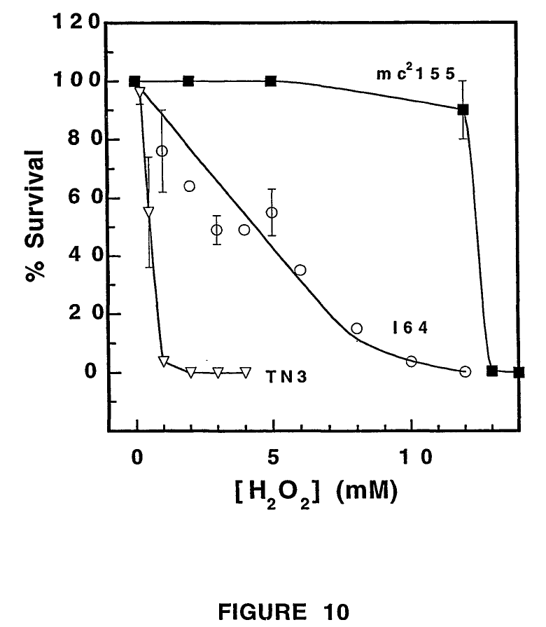

The results of these experiments show that the parent strain (mc² 155) has a high MSH content and exhibits a marked resistance to killing by hydrogen peroxide. By contrast, strain 49, which is blocked in the first step of MSH biosynthesis and produces no detectable MSH, is 12-fold more sensitive to H₂O₂. Partial restoration of MSH biosynthesis in strain 49 through complementation (strain 49::H37Rv A18) reduced the peroxide sensitivity of mutant 49 by a factor of two. Chemical mutant 164, blocked at the ligase biosynthetic enzyme MshC, has a low residual MSH content and is three-fold more sensitive to peroxide than the parent strain. However, transposon mutant TN3, also blocked at MshC but producing no detectable MSH, was >20-fold more sensitive to H₂O₂ than the parent strain (FIG. 10). These results show that blocking MSH biosynthesis before or after the deacetylase step (MshB) produces a dramatic increase in peroxide sensitivity. It logically follows that blocking-MSH biosynthesis at MshB would produce a comparable increase in peroxide sensitivity in mycothiol-producing bacteria.

The sensitivity of strains mc²155, 164, and TN3 to superoxide was also examined (courtesy of Drs. H. Buchmeier and D. Piddington). Superoxide sensitivity was determined according to the method of De Groote, et al. (1997) Proc. Natl. Acad. Sci. 94: 13997-14001 using timed exposure of cells to 250 μM hypoxanthine in the presence of 0.1 unit/ml of xanthine oxidase followed by dilution and plating to determine survival. Under these conditions, strain mc²155 exhibits no significant killing whereas mutant TN3 is >90% killed in 1 hour. By contrast, chemical mutant 164, with a low residual MSH content, exhibited an intermediate level of killing.

Survival of mycothiol-deficient mutant in macrophage. Transposon mutant TN3 was compared with the parent strain for survival in murine macrophages (data courtesy of Drs. N. Buchmeier and D. Piddington). Following the method of De Groote, et al. (1997) Supra, periodate activated, γ-interferon treated murine peritoneal exudate cells were infected with TN3 and mc²155, and extracellular bacteria were washed free of the macrophages with medium. At timed intervals the macrophages were resuspended in PBS and lysed with 0.5% deoxycholate; serial dilutions of the lysate were plated on Middlebrook 7H9 agar for determination of colony forming units. The results of two independent experiments performed on different days (FIG. 11, experiments 1 and 2) show no loss of viability of the parent strain mc²155 in the macrophages over the period of 24 hours; whereas over 60% of the MSH-deficient mutant TN3 was killed by the macrophages within 6 hours. These results suggest that mycobacteria deficient in MSH biosynthesis will exhibit significantly enhanced killing by mammalian macrophages.

While the invention has been described in detail with reference to certain preferred embodiments thereof, it will be understood that modifications and variations are within the spirit and scope of that which is described and claimed.

1. A purified acetyl glucosaminyl inositol deacetylase, characterized as having:
a) an N-terminal region with an amino acid sequence with 40% or more sequence identity to SEQ ID NO:2 and conservative variations thereof,
b) three domains of conservation, wherein two of the domains contain conserved histidine residues, and
c) deacetylase activity against acetyl glucosaminyl inositol.

2. The purified deacetylase of claim 1, wherein the deacetylase hydrolyzes a C2-amide bond of the glucosaminyl inositol moiety.

3. The purified deacetylase of claim 1, wherein the acetyl glucosaminyl inositol is a precursor of mycothiol.

4. The purified deacetylase of claim 1, wherein the acetyl glucoasaminyl inositol is N-acetyl-1-D-myo-inosityl-2-amino-2-deoxy-α-D-glucopyranoside (GlcNAc-Ins).

5. The purified deacetylase of claim 1, wherein the three domains have amino acid sequences selected from the group consisting of SEQ ID NOS: 3, 4, 5, 6, conservative variations thereof, and any combination of two or more thereof.

6. The purified deacetylase of claim 1, wherein the deacetylase is derived from an actinomycetes.

7. The purified deacetylase of claim 6, wherein the deacetylase is derived from M. smegmatis.

8. The purified deacetylase of claim 6, wherein the deacetylase is derived from M. tuberculosis.

9. The purified deacetylase of claim 6, wherein the deacetylase is derived from M. leprae.

10. The purified deacetylase of claim 6, wherein the deacetylase is derived from M. bovis.

11. The purified deacetylase of claim 6, wherein the deacetylase is derived from M. smegmatis, M. tuberculosis, M. leprae, M. bovis, M. intracellulare, M. africanum, M. marinarum. M. chelonai, Corynebacterium diphtheriae, Actinomyces israelii, M. avium complex (MAC), M. ulcerans, M. abscessus, or M. scrofulaceum.

12. The purified deacetylase of claim 6, wherein the bacterium is selected from the group consisting of Streptomyces lincolnensis, Amycolatopsis mediterranei, Amycolatopsis orientalis, Streptomyces lavendulae, Streptomyces coelicolor, Streptomyces rochei and Saccharopolyspora erythraea.

13. The purified deacetylase of claim 1, wherein the deacetylase has amidase activity against acyl glucosaminyl inositols.

14. The purified deacetylase of claim 1, wherein the deacetylase has an amino acid sequence as set forth in SEQ ID NO:1

15. The purified deacetylase of claim 1, wherein the deacetylase is encoded by a polynucleotide comprising a nucleic acid sequence as set forth in SEQ ID NO:7.

16. A purified acetyl glucosaminyl inositol deacetylase, characterized as having:
a) an N-terminal region with an amino acid sequence with at least 40% sequence identity to SEQ ID NO:2,
b) one or more domains of conservation containing conserved metal chelating residues, and
c) deacetylase activity against acetyl glucosaminyl inositol in the presence of metal ion.

17. The purified deacetylase of claim 16, wherein the metal ion is selected from the group consisting of Mn²⁺ and Ni², Cd²⁺, Co²⁺, and Zn²⁺.

18. The purified deacetylase of claim 17 wherein the metal ion is Zn²⁺.

19. The purified deacetylase of claim 16, wherein the metal chelating residues are selected from the group consisting of histidine, aspartic acid and glutamic acid, and combinations thereof.

20. An antibody, or functional fragment thereof, that binds specifically to an deacetylase of claim 1.

21. An isolated polynucleotide that encodes a deacetylase of claim 1.

22. A vector containing a polynucleotide that encodes an deacetylase of claim 1.

23. A cell transformed with a vector of claim 22.

24. A method for identifying an inhibitor of acetyl glucosaminyl inositol deacetylase, said method comprising:
a) contacting a candidate compound with a deacetylase of claim 1 in the presence of an acyl glucosaminyl inositol under suitable conditions and
b) determining the presence or absence of breakdown products of the acyl glucosaminyl inositol indicative of deacetylase activity or amidase activity,
wherein the substantial absence of the deacetylase activity or the amidase activity is indicative of a candidate compound that inhibits activity of the deacetylase.

25. The method of claim 24, wherein the deacetylase is an acetyl glucosaminyl inositol deacetylase.

26. The method of claim 24, wherein a breakdown product is a free amine.

27. The method of claim 26, wherein the free amine is GlcN-Ins.

28. The method of claim 24, wherein a breakdown product is acetate

29. The method of claim 24, wherein a breakdown product is a derivative of cysteine.

30. The method of claim 24, wherein the acyl glucosaminyl inositol is N-acetyl-1-D-myo-inosityl-2-amino-2-deoxy-α-D-glucopyranoside.

31. The method of claim 24, wherein the acyl glucosaminyl inositol is an S-conjugate of mycothiol.

32. The method of claim 31, wherein the S-conjugate of mycothiol is the monobromobimane derivative of mycothiol (MSmB).

33. The method of claim 24, wherein the three domains in the deacetylase that contain conserved histidine residues have amino acid sequences selected from SEQ ID NOs: 3, 5, 6 and any combination of two or more thereof.

34. The method of claim 24, wherein the deacetylase is produced in an actinomycete.

35. The method of claim 34, wherein the actinomycete is M. smegmatis.

36. The method of claim 34, wherein the actinomycete is M. tuberculosis.

37. The method of claim 34, wherein the actinomycete is M. leprae.

38. The method of claim 34, wherein the actinomycete is M. bovis.

39. The method of claim 34, wherein the actinomycete is M. intracellulare, M. africanum, M. marinarum, M. chelonai, Corynebacterium diphtheriae, Actinomyces israelii, M. avium complex (MAC), M. ulcerans, M. abscessus, or M. scrofulaceum.

40. The method of claim 24, wherein candidate compound is a polypeptide, polynucleotide or small molecule.

41. A high throughput screening method for identifying inhibitors of the deacetylase of claim 1, said method comprising:
a) contacting each of a plurality of candidate compounds with a deacetylase of claim 1 in the presence of acyl glucosaminyl inositol under suitable conditions to form a plurality of reaction mixtures and
b) determining the presence or absence of binding to the reaction mixtures of a detectable marker that binds to free amine,
wherein the substantial absence of binding of the marker to a reaction mixture is indicative of a candidate compound that inhibits activity of the deacetylase.

42. The method of claim 41, wherein the plurality of reaction mixtures are formed in the wells of a microtiter plate.

43. The method of claim 41, wherein the detectable marker is colorometric or fluorometric.

44. The method of claim 43 wherein the fluorometric marker is fluorescamine or 6-aminoquinolyl-N-hydroxysuccinimidyl carbamate.

45. An inhibitor of the deacetylase of claim 1 wherein the inhibitor is derived from GlcN-Ins by replacing the amino group therein with a moiety that chelates Zn²⁺, or otherwise binds the enzyme active site of the deacetylase.

46. The inhibitor of claim 45 wherein the inhibitor additionally chelates one or more metal ions metal ions selected from the group consisting of Mn²⁺ and Ni², Cd²⁺, and Co²⁺.

47. A derivative of GlcN-Ins, wherein the derivative contains a reactive residue attached to the amino group that promotes oxidative stress so as to be selectively toxic to a mycothiol-producing actinomycete by being concentrated in the actinomycete.

48. The derivative of claim 47, wherein the reactive residue is a nitroso residue.

49. The derivative of claim 47, wherein the reactive residue is a nitroalkyl residue comprising 1 to 3 carbon atoms.

50. The derivative of claim 47, wherein the reactive residue is a S-nitrosomercaptoalkyl residue comprising 2 to 4 carbon atoms.

51. The derivative of claim 47, wherein the reactive residue is a peroxyalkyl residue comprising 2 to 4 carbon atoms.

52. A live mutant actinomycete, whose genome comprises a disruption in an endogenous acetyl glucosaminyl inositol deacetylase gene, wherein said disruption prevents function of an endogenous acetyl glucosaminyl inositol deacetylase while cell surface proteins and lipids are substantially unaffected, and wherein said disruption results in said mutant actinomycetes exhibiting transient survival in mammalian white blood cells for an immune response-raising period of time.

53. The live mutant actinomycete of claim 52, wherein the period of time is from 1 to 30 days.

54. The live mutant actinomycete of claim 52, wherein the survival of the mutant actinomycetes in mammalian white blood cells does not exceed 30 days.

55. The live mutant actinomycete of claim 52, wherein the mutant actinomycete is derived from a pathogen selected from the group consisting of M. smegmatis, M. tuberculosis, M. leprae, M. bovis, M. intracellulare, M. africanum, M. marinarum, M. chelonai, Corynebacterium diphtheria, Actinomyces israelii, M. avium complex (MAC), M. ulcerans, M. abscessus, and M. scrofulaceum.

56. The live mutant actinomycete of claim 52, wherein the mutant actinomycetes. is derived from M. smegmatis.

57. The live mutant actinomycete of claim 52, wherein the mutant actinomycetes is derived from M. tuberculosis.

58. The live mutant actinomycete of claim 52, wherein the mutant actinomycetes is derived from M. leprae.

59. The live mutant actinomycete of claim 52, wherein the mutant actinomycetes is derived from M. bovis and said mammal is bovine.

60. A method for decreasing the virulence of a pathogenic acetyl glucosaminyl inositol deacetylase-producing bacterium in mammalian cells, said method comprising:
introducing into the bacterium an inhibitor of acetyl glucosaminyl inositol deacetylase activity,
wherein the intracellular presence of the inhibitor decreases activity of the deacetylase, thereby decreasing mycothiol biosynthesis by the bacterium as compared with untreated control bacterium.

61. The method of claim 60, wherein the inhibitor inhibits intracellular production of the deacetylase.

62. The method of claim 60, wherein the inhibitor inhibits intracellular deacetylase activity of the deacetylase.

63. The method of claim 60, wherein the introducing comprises culturing the bacterium in the presence of the inhibitor.

64. The method of claim 60, wherein the inhibitor is an anti-sense oligonucleotide complementary to a target region in a messenger RNA that encodes a polypeptide having an amino acid sequence segment with 40% or more sequence identity to the amino acid sequence of SEQ ID NO:2 or at least one amino acid sequence selected from the group consisting of SEQ ID NOS: 3, 4, 5, 6 and conservative variations thereof.

65. The method of claim 60, wherein the inhibitor is an anti-sense oligonucleotide that hybridizes under intracellular conditions with a messenger RNA that encodes a polypeptide having an N-terminal amino acid sequence as set forth in SEQ ID NO:2.

66. The method of claim 60, wherein the bacterium is an actinomycete and the inhibitor inhibits intracellular production of mycothiol.

67. The method of claim 60, wherein the bacterium is selected from the group consisting of the pathogenic bacteria M. smegmatis, M. tuberculosis, M. leprae, M. bovis, M. intracellulare, M. africanum, M. marinarum, M. chelonai, Corynebacterium diphtheria, Actinomyces israelii, M. avium complex (MAC), M. ulcerans, M abscessus, and M. scrofulaceum.

68. The method of claim 60, wherein the bacterium is an actinomycete.

69. A method for inhibiting growth of an acetyl glucosaminyl inositol-producing bacterium in a mammal, said method comprising administering to the mammal an effective amount of an inhibitor of intracellular acetyl glucosaminyl inositol deacetylase, thereby inhibiting growth of the bacterium in the mammal.

70. The method of claim 69, wherein the inhibitor is derived from GlcN-Ins by replacing the amino group therein with a moiety that chelates a metal ion selected from the group consisting of Mn²⁺ and Ni², Cd²⁺, Co²⁺, and Zn²⁺, or otherwise binds the enzyme active site in the deacetylase.

71. The method of claim 70, wherein the moiety is ClCH₂CONH—.

72. The method of claim 70 wherein the moiety is HONHCONH—.

73. The method of claim 70, wherein the moiety is HONHCOCH₂—.

74. The method of claim 70, wherein the moiety is HOPO(CH₃)NH—.

75. The method of claim 70, wherein the moiety is HOPO(CH₃)ₙCH₂—; wherein n=1-5.

76. The method of claim 70 wherein the moiety is HSCH₂(CH₂)ₙNH—; wherein n=2-5.

77. The method of claim 70 wherein the moiety is HS(CH₂)ₙCONH—; wherein n=1-3.

78. The method of claim 69 wherein the acetyl glucosaminyl inositol-producing bacterium is a mycothiol-producing bacterium.

79. A process for preparation of 1-D-myo-inosityl-2-amino-2-deoxy-α-D-glucopyranoside (GlcN-Ins), said method comprising:
contacting an acyl glucosaminyl inositol with an deacetylase of claim 1 under suitable conditions so as to hydrolyze the amide bond therein, and
obtaining the GlcN-Ins.

80. A method for determination of acetyl glucosaminyl inositol (GlcNAc-Ins) in a sample, said method comprising:
a) contacting a sample containing GlcNAc-Ins with a deacetylase of claim 1 under suitable conditions and
b) determining the amount of GlcN-Ins produced, wherein the amount of GlcN-Ins produced is a measure of the GlcNAc-Ins in the sample.

81. The method of claim 80, wherein the amount of GlcN-Ins is determined by HPLC after labeling thereof with a fluorometric or calorimetric reagent.

82. The method of claim 81, wherein the fluorometric reagent is fluorescamine.

83. The method of claim 81, wherein the fluorometric reagent is 6-aminoquinolyl-N-hydroxysuccinimidyl carbamate.
